SEQUENCE LISTING (25 sequences)

SEQ ID NO: 1 - protein, 472 aa, Artificial
  Recombinant antibody heavy chain polypeptide sequence
METDTLLLWVLLLWVPGSTGDQLQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARTPTVTGDWFDPWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, Artificial
  Recombinant antibody light chain polypeptide sequence
MRVPAQLLGLLLLWLPGARCGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 384 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 121 aa, Artificial
  Immunoglobulin F5111 heavy chain variable domain polypeptide sequence
QLQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARTPTVTGDWFDPWGRGTLVTVSS

SEQ ID NO: 5 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 21 aa, Artificial
  Signal sequence
METDTLLLWVLLLWVPGSTGD

SEQ ID NO: 7 - protein, 20 aa, Artificial
  Signal sequence
MRVPAQLLGLLLLWLPGARC

SEQ ID NO: 8 - protein, 20 aa, Artificial
  Signal sequence
MYRMQLLSCIALSLALVTNS

SEQ ID NO: 9 - protein, 133 aa, Homo sapiens [sequence omitted]

SEQ ID NO: 10 - protein, 110 aa, Artificial
  Immunoglobulin F5111 light chain variable domain polypeptide sequence
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVL

SEQ ID NO: 11 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 10 aa, Artificial
  Polypeptide linker
GGGGSGGGGS

SEQ ID NO: 13 - protein, 15 aa, Artificial
  Polypeptide linker
GGGGSGGGGSGGGGS

SEQ ID NO: 14 - protein, 20 aa, Artificial
  Polypeptide linker
GGGGSGGGGSGGGGSGGGGS

SEQ ID NO: 15 - protein, 25 aa, Artificial
  Polypeptide linker
GGGGSGGGGSGGGGSGGGGSGGGGS

SEQ ID NO: 16 - protein, 30 aa, Artificial
  Polypeptide linker
GGGGSGGGGSGGGGSGGGGSGGGGSGGGGS

SEQ ID NO: 17 - protein, 35 aa, Artificial
  Polypeptide linker
GGGGSGGGGSGGGGSGGGGSGGGGSGGGGSGGGGS

SEQ ID NO: 18 - protein, 40 aa, Artificial
  Polypeptide linker
GGGGSGGGGSGGGGSGGGGSGGGGSGGGGSGGGGSGGGGS

SEQ ID NO: 19 - protein, 45 aa, Artificial
  Polypeptide linker
GGGGSGGGGSGGGGSGGGGSGGGGSGGGGSGGGGSGGGGSGGGGS

SEQ ID NO: 20 - protein, 50 aa, Artificial
  Polypeptide linker
GGGGSGGGGSGGGGSGGGGSGGGGSGGGGSGGGGSGGGGSGGGGSGGGGS

SEQ ID NO: 21 - protein, 55 aa, Artificial
  Polypeptide linker
GGGGSGGGGSGGGGSGGGGSGGGGSGGGGSGGGGSGGGGSGGGGSGGGGSGGGGS

SEQ ID NO: 22 - protein, 60 aa, Artificial
  Polypeptide linker
GGGGSGGGGSGGGGSGGGGSGGGGSGGGGSGGGGSGGGGSGGGGSGGGGSGGGGSGGGGS

SEQ ID NO: 23 - protein, 14 aa, Artificial
  Biotin acceptor peptide
LNDIFEAQKIEWHE

SEQ ID NO: 24 - protein, 394 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 404 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2022/0242946 A1
(43) Pub. Date: Aug. 4, 2022

(54) METHODS AND MATERIALS FOR TARGETED EXPANSION OF REGULATORY T CELLS

(71) Applicant: The Johns Hopkins University, Baltimore, MD (US)

(72) Inventors: Jamie Spangler, Baltimore, MD (US); Derek VanDyke, Baltimore, MD (US)

(21) Appl. No.: 17/622,504

(22) PCT Filed: Jun. 26, 2020
(86) PCT No.: PCT/US2020/039854

§ 371 Date: Dec. 23, 2021

Related U.S. Application Data
(60) Provisional application No. 62/867,012, filed on Jun. 26, 2019.

(51) Int. Cl. C07K 16/24; A61K 38/20; C07K 14/55; C07K 14/715; A61P 37/06

(52) U.S. Cl. CPC C07K 16/246; A61K 38/2013; A61K 2039/505; C07K 14/7155; A61P 37/06; C07K 14/55

(57) ABSTRACT

This document relates to methods and materials for targeted expansion of regulatory T cells (T_{RegS}). For example, one or more single-chain antibody/cytokine fusion proteins (immunocytokines) that can bind to a heterotrimeric receptor including an interleukin-2 receptor-α(IL-2Rα) polypeptide, an interleukin-2 receptor-β(IL-2Rβ) polypeptide, and a common gamma chain (γc) polypeptide (e.g., an IL-2Rα/IL-2Rβ/γc polypeptide complex) can be administered to a mammal to stimulate T_{RegS} within the mammal to reduce or eliminate an immune response in that mammal. In some cases, methods and materials that can be used to treat a mammal having a condition that can benefit from reducing or eliminating an immune response within the mammal are provided. For example, one or more single-chain immunocytokines that can bind to an IL-2Rα/IL-2Rβ/γc polypeptide complex can be administered to a mammal having a condition that can benefit from reducing or eliminating an immune response to treat the mammal.

### CROSS-REFERENCE TO RELATED APPLICATIONS

This application claims the benefit of U.S. patent application Ser. No. 62/867,012, filed on Jun. 26, 2019. The disclosure of the prior application is considered part of (and is incorporated by reference in) the disclosure of this application.

### STATEMENT REGARDING FEDERAL FUNDING

This invention was made with government support under W81XWH-18-1-0735 awarded by the U.S. Department of Defense. The government has certain rights in the invention.

### BACKGROUND

### 1. Technical Field

This document relates to methods and materials for targeted expansion of regulatory T cells (T_{RegS}). For example, a composition containing one or more amino acid chains (e.g., one or more single-chain antibody/cytokine fusion proteins (immunocytokines)) that can bind to a heterotrimeric receptor including an interleukin-2 receptor-α (IL-2Rα) polypeptide, an interleukin-2 receptor-β (IL-2Rβ) polypeptide, and a common gamma chain (γc) polypeptide (e.g., an IL-βRα/IL-2Rβ/γc polypeptide complex) can be administered to a mammal to stimulate T_{RegS} within the mammal to reduce or eliminate an immune response (e.g., an autoimmune response) in that mammal. In some cases, methods and materials provided herein can be used to treat a mammal having a condition that can benefit from reducing or eliminating an immune response within the mammal (e.g., an autoimmune disease and/or transplant rejection). For example, a composition containing one or more single-chain immunocytokines that can bind to an IL-2Rα/IL-βRβ/γc polypeptide complex can be administered to a mammal having a condition that can benefit from reducing or eliminating an immune response to treat the mammal.

### 2. Background Information

IL-2 is a multi-functional cytokine that orchestrates the differentiation, proliferation, survival, and activity of immune cells. Low-dose IL-2 treatment preferentially stimulates polyclonal expansion of TRe_{g}s over immune effector cells (Effs; Boyman et al., Nat Rev Immunol. 12(3):180-190 (2012); and Liao et al., Immunity. 38(1):13-25 (2013)). Preclinical and clinical work demonstrates that low-dose IL-2 can promote TRe_{g} expansion; however, IL-2 can also expand Effs (e.g., natural killer (NK) cells, natural killer T (NKT) cells, CD4⁺ effector T cells, and CD8⁺ effector T cells), which leads to undesirable off-target effects and toxicities (Boyman et al., Nat Rev Immunol. 12(3):180-190 (2012); and Klatzmann et al., Nat Rev Immunol. 15(5):283-294 (2015)).

### SUMMARY

IL-2 activates cell signaling through either a high-affinity (K_{D}≈10 pM) heterotrimeric receptor consisting of the IL-2Rα, IL-2Rβ, and γc chains, or an intermediate-affinity (K_{D}≈1 nM) heterodimeric receptor consisting of only the IL-2Rβ and γc chains. Consequently, IL-2 responsiveness is determined by the IL-2Rα subunit, which is highly expressed on T_{RegS}, but virtually absent from naïve Effs, rendering T_{RegS} 100-fold more sensitive to IL-2 (see, e.g., Boyman et al., Nat Rev Immunol. 12(3):180-90 (2012); Malek, Annu Rev Immunol. 26:45379 (2008); and Spangler et al., Annu Rev Immunol. 33:139-67 (2015)). The ability to isolate and selectively tune the immunosuppressive activities of IL-2 would represent a transformative advance for immunotherapeutic development, with important implications for autoimmune disease and transplantation medicine.

This document provides methods and materials for targeted expansion of T_{RegS}. For example, provided herein are single-chain immunocytokines that can bind to an IL-2Rα/IL-2Rβ/γyc polypeptide complex. In some cases, a single-chain immunocytokine that can bind to an IL-2Rα/IL-2Rβ/γc polypeptide complex can include (e.g., can be designed to include) an immunoglobulin heavy chain (HC), an IL-2 polypeptide (or fragment thereof) that can bind an IL-2Rα/IL-2Rβ/γc polypeptide complex, and an immunoglobulin light chain (LC). Also provided herein are methods for making and using single-chain immunocytokines that can bind to an IL-2Rα/IL-2Rβ/γc polypeptide complex. For example, a composition containing one or more single-chain immunocytokines that can bind to an IL-2Rα/IL-2Rβ/γc polypeptide complex can be administered to a mammal in need thereof (e.g., a mammal having a condition that can benefit from reducing or eliminating an immune response within the mammal such as an autoimmune disease and/or transplant rejection) to treat the mammal. In some cases, a composition containing one or more single-chain immunocytokines that can bind to an IL-2Rα/IL-2Rβ/γc polypeptide complex can be administered to a mammal to stimulate T_{RegS} within the mammal (e.g., to reduce or eliminate an immune response such as an autoimmune response in that mammal). For example, a composition containing one or more single-chain immunocytokines that can bind to an IL-2Rα/IL-2Rβ/γc polypeptide complex can be administered to a mammal having an autoimmune disease to treat the mammal. For example, a composition containing one or more single-chain immunocytokines that can bind to an IL-2Rα/IL-2Rβ/γc polypeptide complex can be administered to a mammal having, or at risk of developing, transplant rejection to treat the mammal.

As demonstrated herein, a single-chain immunocytokine engineered to bind to an IL-2Rα/IL-2Rβ/γc polypeptide complex can specifically stimulate (e.g., expand) T_{RegS}in vivo, and can suppress pathogenic autoimmunity in vivo. The ability to stimulate immune T_{RegS} (e.g., but not Effs) provides unique and unrealized targeted cytokine therapies that can safely and selectively reduce or eliminate pathogenic autoimmunity and/or transplant rejection in a mammal (e.g., a human), and can be used to treat a mammal having an autoimmune disease and/or having, or at risk of developing, transplant rejection.

In general, one aspect of this document features single-chain immunocytokines including (a) an immunoglobulin heavy chain; (b) an IL-2 polypeptide, where the IL-2 polypeptide can bind to an IL-2Rα/IL-2Rβ/γc polypeptide complex; and (c) an immunoglobulin light chain; where the single-chain immunocytokine binds to the IL-2Rα/IL-2Rβ/γc polypeptide complex. The immunoglobulin heavy chain can include a variable domain having at least 80% identity to an amino acid sequence set forth in SEQ ID NO:4. The immunoglobulin heavy chain can include a variable domain having an amino acid sequence set forth in SEQ ID NO:4. The immunoglobulin heavy chain can include a y heavy chain constant domain. The γ heavy chain constant domain can have at least 70% identity to an amino acid sequence set forth in SEQ ID NO:5. The immunoglobulin heavy chain can include a constant domain having an amino acid sequence set forth in SEQ ID NO:5. The immunoglobulin heavy chain can include a signal sequence. The signal sequence can include an amino acid sequence set forth in SEQ ID NO:6. The immunoglobulin heavy chain can include an amino acid sequence set forth in SEQ ID NO:1. The IL-2 polypeptide can include an amino acid sequence having at least 80% identity to an amino acid sequence set forth in SEQ ID NO:9. The IL-2 polypeptide can include an amino acid sequence set forth in SEQ ID NO:9. The immunoglobulin light chain can include a variable domain having at least 80% identity to an amino acid sequence set forth in SEQ ID NO: 10. The immunoglobulin light chain can include a variable domain having an amino acid sequence set forth in SEQ ID NO:10. The immunoglobulin light chain can include a lambda (λ) light chain constant domain. The λ light chain constant domain can have at least 70% identity to an amino acid sequence set forth in SEQ ID NO:11. The immunoglobulin light chain can include a constant domain having an amino acid sequence set forth in SEQ ID NO:11. The immunoglobulin light chain can include a signal sequence. The signal sequence can include an amino acid sequence set forth in SEQ ID NO:7. The immunoglobulin light chain can include an amino acid sequence set forth in SEQ ID NO:2. The IL-2 polypeptide and the immunoglobulin light chain can be a fusion polypeptide. The IL-2 polypeptide can include an amino acid sequence having at least 80% identity to an amino acid sequence set forth in SEQ ID NO:9. The IL-2 polypeptide can include an amino acid sequence set forth in SEQ ID NO:9. The immunoglobulin light chain can include a variable domain having at least 80% identity to an amino acid sequence set forth in SEQ ID NO:10. The immunoglobulin light chain can include a variable domain having an amino acid sequence set forth in SEQ ID NO:10. The immunoglobulin light chain can include a λ light chain constant domain. The λ light chain constant domain can have at least 70% identity to an amino acid sequence set forth in SEQ ID NO:11. The immunoglobulin light chain can include a constant domain having an amino acid sequence set forth in SEQ ID NO:11. The IL-2 polypeptide and the immunoglobulin light chain can be fused via a linker. The linker can be a peptide linker that can include from 10 to 60 amino acids. The linker can be a (Gly₄Ser)₃, a (Gly₄Ser)₅, or a (Gly₄Ser)₇ linker. The immunoglobulin light chain can include a signal sequence. The signal sequence can include an amino acid sequence set forth in SEQ ID NO:8. The immunoglobulin light chain can include an amino acid sequence set forth in SEQ ID NO:3, SEQ ID NO:24, or SEQ ID NO:25. The single-chain immunocytokine can have a half-life of from about 5 minutes to about 6 months. The single-chain immunocytokine can have an affinity for an IL-2Rα polypeptide of from about 10 nM K_{D} to about 1 pM K_{D}. The single-chain immunocytokine can have an affinity for an IL-2Rβ polypeptide of greater than about 300 nM K_{D}. In some cases, the single-chain immunocytokine can bind to a human IL-2Rα/IL-2Rβ/γc polypeptide complex. In some cases, the single-chain immunocytokine does not bind to a non-human IL-2Rα/IL-2Rβ/γc polypeptide complex.

In another aspect, this document features nucleic acids encoding a single-chain immunocytokine including (a) an immunoglobulin heavy chain; (b) an IL-2 polypeptide, where the IL-2 polypeptide can bind to an IL-2Rα/IL-2Rβ/γc polypeptide complex; and (c) an immunoglobulin light chain; where the single-chain immunocytokine binds to the IL-2Rα/IL-2Rβ/γc polypeptide complex. The nucleic acid can include a first nucleic acid and a second nucleic acid, where said first nucleic acid can encode an immunoglobulin heavy chain, and where the second nucleic acid can encode the IL-2 polypeptide fused to the immunoglobulin light chain.

In another aspect, this document features methods for treating a mammal having an autoimmune disease. The methods can include, or consist essentially of, administering a composition comprising one or more single-chain immunocytokines including (a) an immunoglobulin heavy chain; (b) an IL-2 polypeptide, where the IL-2 polypeptide can bind to an IL-2Rα/IL-2Rβ/γc polypeptide complex; and (c) an immunoglobulin light chain; where the single-chain immunocytokine binds to the IL-2Rα/IL-2Rβ/γc polypeptide complex; or a composition comprising nucleic acid encoding a single-chain immunocytokine including (a) an immunoglobulin heavy chain; (b) an IL-2 polypeptide, where the IL-2 polypeptide can bind to an IL-2Rα/IL-2Rβ/γc polypeptide complex; and (c) an immunoglobulin light chain; where the single-chain immunocytokine binds to the IL-2Rα/IL-2Rβ/γc polypeptide complex to a mammal having an autoimmune disease. The mammal can be a human. The autoimmune disease can be type 1 diabetes, multiple sclerosis, Chron's disease, ulcerative colitis, psoriasis, graft-versus-host disease, Guillain-Barre syndrome, lupus, rheumatoid arthritis, chronic inflammatory demyelinating polyneuropathy, Hashimoto Thyroiditis, Celiac disease, Addison disease, autoimmune hepatitis, antiphospholipid syndrome, or Graves disease. The method also can include administering one or more autoimmune disease treatments to the mammal under conditions wherein number of autoantibodies present in the mammal is reduced. The method does not substantially activate effector T cells.

In another aspect, this document features methods for stimulating regulatory T cells in a mammal. The methods can include, or consist essentially of, administering a composition comprising one or more single-chain immunocytokines including (a) an immunoglobulin heavy chain; (b) an IL-2 polypeptide, where the IL-2 polypeptide can bind to an IL-2Rα/IL-2Rβ/γc polypeptide complex; and (c) an immunoglobulin light chain; where the single-chain immunocytokine binds to the IL-2Rα/IL-2Rβ/γc polypeptide complex; or a composition comprising nucleic acid encoding a single-chain immunocytokine including (a) an immunoglobulin heavy chain; (b) an IL-2 polypeptide, where the IL-2 polypeptide can bind to an IL-2Rα/IL-2β/γc polypeptide complex; and (c) an immunoglobulin light chain; where the single-chain immunocytokine binds to the IL-2Rα/IL 2Rβ/γc polypeptide complex to a mammal. The mammal can be a human. The method does not substantially activate effector T cells.

In another aspect, this document features methods for treating a mammal having a transplant rejection. The methods can include, or consist essentially of, administering a composition comprising one or more single-chain immunocytokines including (a) an immunoglobulin heavy chain; (b) an IL-2 polypeptide, where the IL-2 polypeptide can bind to an IL-2Rα/IL 2Rβ/γc polypeptide complex; and (c) an immunoglobulin light chain; where the single-chain immunocytokine binds to the IL-2Rα/IL 2Rβ/γc polypeptide complex; or a composition comprising nucleic acid encoding a single-chain immunocytokine including (a) an immunoglobulin heavy chain; (b) an IL-2 polypeptide, where the IL-2 polypeptide can bind to an IL-2Rα/IL 2Rβ/γc polypeptide complex; and (c) an immunoglobulin light chain; where the single-chain immunocytokine binds to the IL-2Rα/IL 2Rβ/γc polypeptide complex to a mammal having transplant rejection. The mammal can be a human. The transplant rejection can be a rejection of an allogeneic transplant or a rejection of an autologous transplant. The does not substantially activate effector T cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic of the design of the IL-2/F5111 single chain fusion protein (immunocytokine). Human IL-2 is fused to the N-terminus of the F5111 antibody light chain.

FIG. 2A is a graph illustrating FPLC traces of recombinant F5111 antibody (left panel) and F5111 immunocytokine (IC) LN15 (right panel). LN15 refers to a 15-amino acid linker between the C-terminus of human IL-2 and the N-terminus of the F5111 antibody light chain. Pooled fractions are indicated by a solid line. FIG. 2B is an image of non-reducing and reducing SDS-PAGE analyses of purified F5111 antibody and F5111 IC LN15.

FIG. 3 is a graph showing that F5111 antibody binds human but not mouse IL-2 cytokine. Yeast surface binding of F5111 antibody to human IL-2 (hIL-2, solid line) or mouse IL-2 (mIL-2, dashed line) is shown, as measured by flow cytometry.

FIG. 4A is a graph depicting binding of the F5111 antibody and IC to yeast surface-displayed hIL-2, as measured by flow cytometry. FIG. 4B is a graph showing binding of purified F5111 antibody, hIL-2/F5111 complex, and F5111 IC LN15 to immobilized hIL-2, as measured by bio-layer interferometry. An irrelevant protein (the monoclonal antibody trastuzumab) was used as a negative control.

FIG. 5A is a graph showing bio-layer interferometry binding titrations of hIL-2, hIL-2/F5111 complex, and F5111 IC LN15 against immobilized IL-2Rα. An irrelevant protein (the monoclonal antibody trastuzumab) was used as a negative control. FIG. 5B is a graph showing bio-layer interferometry binding titrations of hIL-2, hIL-2/F5111 complex, and F5111 IC LN15 against immobilized IL-2Rβ. An irrelevant protein (the monoclonal antibody trastuzumab) was used as a negative control.

FIG. 6 includes schematics and graphs illustrating that F5111 IC LN15 selectively activates IL-2Rα⁺ cells. STATS activation in response to IL-2, IL-2/F5111 complex, or F5111 IC LN15 on YT-1 human natural killer (NK) cells with (FIG. 6A) or without (FIG. 6B) IL-2Rα is shown, as measured by flow cytometry.

FIG. 7 shows that F5111 IC LN25 and LN35 were produced in HEK293 cells and purified using size exclusion chromatography (SEC). FIG. 7A is a graph showing the SEC trace for the F5111 IC LN35. It is expected that Peak 1 (P1) and Peak 2 (P2) contain higher order oligomeric structures, whereas Peak 3 (P3) contains the monomeric F5111 IC LN35. Therefore, P3 was used for all subsequent experiments, and F5111 IC LN25 and F5111 IC LN35 refer to the pooled P3 fraction unless otherwise specified. FIG. 7B is a graph showing SEC comparison of F5111 IC LN15, F5111 IC LN25, and F5111 IC LN35. FIG. 7C is an image of SDS-PAGE analysis of F5111 IC LN35 P3 under non-reducing and reducing conditions.

FIG. 8 shows STAT5 activation in response to various IL-2 treatments on IL-2Rα⁺ and IL-2Rα⁻ YT-1 human NK cells. STAT5 activation in response to IL-2, IL-2/F5111 complex, or F5111 IC variants on YT-1 cells with (FIG. 8A) or without (FIG. 8B) IL-2Ra is shown, as measured by flow cytometry.

FIG. 9 shows binding of hIL-2 cytokine/receptor proteins, hIL-2/F5111 complex, and F5111 IC variants to hIL-2 and hIL-2 receptor subunits. FIG. 9A is a graph showing binding of purified F5111 antibody, F5111/hIL-2 complex, and F5111 IC variants to immobilized hIL-2, as measured by bio-layer interferometry. FIG. 9B illustrates binding of purified F5111 antibody, F5111/hIL-2 complex, and F5111 IC variants to immobilized hIL-2Rα, as measured by bio-layer interferometry. FIG. 9C illustrates binding of purified F5111 antibody, F5111/hIL-2 complex, and F5111 IC variants to immobilized hIL-2Rβ, as measured by bio-layer interferometry.

FIG. 10 shows STATS activation in response to hIL-2, hIL-2/F5111 complex, and F5111 IC variants on different immune cell subsets of human peripheral blood mononuclear cells (PBMCs) isolated from whole blood. FIG. 10A shows STATS activation on CD3⁺CD8⁺cells (CD8⁺effector T cells), FIG. 10B shows STATS activation on CD3⁺CD4⁺CD25^{High}FOXP3^{High} cells (T_{Reg} cells), and FIG. 10C shows STATS activation on CD3⁺CD4⁺CD25^{High}FOXP3^{high} cells (CD4⁺effector T cells).

FIG. 11 shows a sequence (SEQ ID NO:1) of an exemplary recombinant antibody heavy chain (corresponding to F5111 antibody) that includes a signal sequence (bold), a F5111 V_{H} (italic), and a human IgG1 CH1, CH2, and CH3 (bold and italic).

FIG. 12 shows a sequence (SEQ ID NO:2) of an exemplary recombinant antibody light chain (corresponding to F5111 antibody) that includes a signal sequence (bold), a F5111 V_{L} (italic), and a Lambda CL (bold and italic).

FIG. 13 shows a sequence (SEQ ID NO:3) of an exemplary immunocytokine light chain (corresponding to F5111 IC LN15) that includes a signal sequence (bold), a hIL-2 (plain text), a linker (underlined), a F5111 V_{L}(italic), and a Lambda C_{L}(bold and italic).

FIG. 14 shows a sequence (SEQ ID NO:24) of an exemplary immunocytokine light chain (corresponding to F5111 IC LN25) that includes a signal sequence (bold), a hIL-2 (plain text), a linker (underlined), a F5111 V_{L}(italic), and a Lambda C_{L} (bold and italic).

FIG. 15 shows a sequence (SEQ ID NO:25) of an exemplary immunocytokine light chain (corresponding to F5111 IC LN35) that includes a signal sequence (bold), a hIL-2 (plain text), a linker (underlined), a F5111 V_{L}(italic), and a Lambda C_{L} (bold and italic).

### DETAILED DESCRIPTION

This document provides methods and materials for targeted expansion of T_{RegS}. For example, provided herein are single-chain immunocytokines that can bind to an IL-2Rα/IL-2Rβ/γc polypeptide complex. In some cases, a single-chain immunocytokine that can bind to an IL-2Rα/IL-2Rβ/γc polypeptide complex can include (e.g., can be designed to include) an immunoglobulin heavy chain, an IL-2 polypeptide (or fragment thereof) that can bind an IL-2Rα/IL-2Rβ/γc polypeptide complex, and an immunoglobulin light chain. Also provided herein are methods for making and using single-chain immunocytokines that can bind to an IL-2Rα/IL-2Rβ/γc polypeptide complex. For example, a composition containing one or more single-chain immunocytokines that can bind to an IL-2Rα/IL-2Rβ/γc polypeptide complex can be administered to a mammal (e.g., a human) in need thereof (e.g., a mammal having a condition that can benefit from reducing or eliminating an immune response within the mammal such as an autoimmune disease and/or transplant rejection) to treat the mammal. In some cases, a composition containing one or more single-chain immunocytokines that can bind to an IL-2Rα/IL-2Rβ/γc polypeptide complex can be administered to a mammal to stimulate T_{RegS} within the mammal (e.g., to reduce or eliminate an immune response such as an autoimmune response in that mammal). For example, a composition containing one or more single-chain immunocytokines that can bind to IL-2Rα/IL-2Rβ/γc polypeptide complex can be administered to a mammal having an autoimmune disease to treat the mammal. For example, a composition containing one or more single-chain immunocytokines that can bind to an IL-2Rα/IL-2Rβ/γc polypeptide complex can be administered to a mammal having, or at risk of developing, transplant rejection to treat the mammal. As used herein, a single-chain immunocytokine described herein (e.g., a single-chain immunocytokine that can bind an IL-2Rα/IL-2Rβ/γc polypeptide complex) is a fusion protein that includes a cytokine fused (e.g., genetically fused) to antibody or a fragment thereof (e.g., a cytokine/antibody fusion protein). In some cases, a single-chain immunocytokine described herein can include a cytokine fused to an anti-cytokine antibody or a fragment thereof (e.g., an anti-IL-2 antibody or a fragment thereof). In some cases, a single-chain immunocytokine described herein can include a cytokine that is fused to an antibody such that the cytokine and antibody bind intramolecularly within the immunocytokine. In some cases, a single-chain immunocytokine described herein can include a cytokine that is fused to one or more ends of an antibody (e.g., the N- or C-terminus of an antibody heavy chain and/or the N- or C-terminus of an antibody light chain). For example, a single-chain immunocytokine can be an amino acid chain that includes (e.g., is designed to include) an immunoglobulin heavy chain, an IL-2 polypeptide (or fragment thereof) that can bind an IL-2Rα/IL-2Rβ/γc polypeptide complex, and an immunoglobulin light chain. In some cases, a single-chain immunocytokine described herein can be a fusion polypeptide that includes a cytokine fused to at least a portion (e.g., an immunoglobulin heavy chain and/or an immunoglobulin light chain) of an anti-cytokine antibody. For example, a single-chain immunocytokine described herein can be a fusion polypeptide that includes an immunoglobulin heavy chain (e.g., an immunoglobulin heavy chain from an anti-cytokine antibody) fused to an IL-2 polypeptide (or fragment thereof) that can bind an IL-2Rα/IL-2Rβ/γc polypeptide complex fused to an immunoglobulin light chain (e.g., an immunoglobulin light chain from an anti-cytokine antibody).

A single-chain immunocytokine described herein (e.g., a single-chain immunocytokine that can bind to an IL-2Rα/IL-2Rβ/γc polypeptide complex) can bind to an IL-2Rα/IL-2Rβ/γc polypeptide complex from any appropriate source (e.g., from any appropriate mammal such as a human or a mouse). In some cases, IL-2 polypeptide (or fragment thereof) that can bind an IL-2Rα/IL 2Rβ/γc polypeptide complex can bind to a human IL-2Rα/IL-2Rβ/γc polypeptide complex. In some cases, where an IL-2 polypeptide (or fragment thereof) that can bind an IL-2Rα/IL 2Rβ/γc polypeptide complex binds to an IL-2Rα/IL 2Rβ/γc polypeptide complex from a first species of mammal, the IL-2 polypeptide (or fragment thereof) that can bind an IL-2Rα/IL-2Rβ/γc polypeptide complex does not cross-react with an IL-2Rα/IL 2Rβ/γc polypeptide complex from a second species of mammal. For example, when an IL-2 polypeptide (or fragment thereof) that can bind an IL-2Rα/IL 2Rβ/γc polypeptide complex binds to a human IL-2Rα/IL-2Rβ/γc polypeptide complex, the IL-2 polypeptide (or fragment thereof) that can bind an IL-2Rα/IL-2Rβ/γc polypeptide complex does not cross-react with an IL-2Rα/IL 2Rβ/γc polypeptide complex from a non-human species (e.g., a mouse IL-2Rα/IL 2Rβ/γc polypeptide complex).

A single-chain immunocytokine described herein (e.g., a single-chain immunocytokine that can bind to an IL-2Rα/IL 2Rβ/γc polypeptide complex) can include any appropriate immunoglobulin (Ig) heavy chain. An immunoglobulin heavy chain can be from any appropriate isotype immunoglobulin (e.g., a IgA immunoglobulin, a IgD immunoglobulin, a IgE immunoglobulin, a IgG immunoglobulin, and a IgM immunoglobulin). In some cases, an immunoglobulin heavy chain can be an IgG heavy chain (e.g., an IgG1 heavy chain). An immunoglobulin heavy chain can be from any appropriate class of immunoglobulin (e.g., γ, σ, α, μ, and ε). An immunoglobulin heavy chain can have any appropriate heavy chain variable domain (V_{H}). An immunoglobulin heavy chain can have any appropriate heavy chain constant domains (C_{H}). In some cases, an immunoglobulin heavy chain can be an immunoglobulin having three constant domains (e.g., C_{H}1, C_{H}2, and C_{H}3) such as a γ heavy chain, an α heavy chain, or a δ heavy chain. In some cases, an immunoglobulin heavy chain can be an immunoglobulin having four constant domains (e.g., C_{H}1, C_{H}2, C_{H}3, and C_{H}4) such as a μ heavy chain or a c heavy chain. An immunoglobulin heavy chain can be from any appropriate immunoglobulin. In some cases, the immunoglobulin heavy chain variable domain and the immunoglobulin heavy chain constant domains can be from the same immunoglobulin. In some cases, the immunoglobulin heavy chain variable domain and the immunoglobulin heavy chain constant domains can be from different immunoglobulins. In some cases, the immunoglobulin heavy chain variable domain and/or the immunoglobulin heavy chain constant domains can be from a naturally occurring immunoglobulin (e.g., can be derived from a naturally occurring immunoglobulin). In some cases, the immunoglobulin heavy chain variable domain and/or the immunoglobulin heavy chain constant domains can be synthetic. Examples of immunoglobulins whose heavy chain variable domain and/or the immunoglobulin heavy chain constant domains can be used in a single-chain immunocytokine described herein include, without limitation, monoclonal antibody F5111 (referred to herein as “F5111”) heavy chains, monoclonal antibody F5111.4 heavy chains, monoclonal antibody F5111.7 heavy chains, monoclonal antibody

F5111.8 heavy chains, and monoclonal antibody F5111.2 heavy chains. In some cases, immunoglobulins whose heavy chain variable domains and/or heavy chain constant domains can be used in a single-chain immunocytokine described herein can be as described elsewhere (see, e.g., Trotta et al., Nat Med. 24(7):10051014 (2018)). An immunoglobulin heavy chain can include any appropriate sequence (e.g., amino acid sequence). In some cases, an immunoglobulin heavy chain variable domain can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:4. For example, a single-chain immunocytokine described herein can include an immunoglobulin heavy chain variable domain having the amino acid sequence set forth in SEQ ID NO:4. In some cases, an immunoglobulin heavy chain constant domain can include an amino acid sequence having at least about 70% identity (e.g., about 75%, about 80%, about 85%, about 88%, about 90%, about 93%, about 95%, about 97%, about, 8%, about 99%, or 100% sequence identity) to the amino acid sequence set forth in SEQ ID NO:5. For example, a single-chain immunocytokine described herein can include an immunoglobulin heavy chain constant domain having the amino acid sequence set forth in SEQ ID NO:5. In some cases, an immunoglobulin heavy chain also can include a signal sequence. A signal sequence can be any appropriate signal sequence (e.g., SEQ ID NO:6 and SEQ ID NO:7). For example, a single-chain immunocytokine described herein can include an immunoglobulin heavy chain having a signal sequence with the amino acid sequence set forth in SEQ ID NO:6.

An exemplary immunoglobulin heavy chain that can be used in a single-chain immunocytokine described herein (e.g., a single-chain immunocytokine that can bind to an IL-2Rα/IL-2Rβ/γc polypeptide complex) is set forth in SEQ ID NO:1, For example, an immunoglobulin heavy chain that can be used in a single-chain immunocytokine described herein can include a signal sequence, a variable domain from a F5111 antibody, and an IgG1 constant domain. For example, an immunoglobulin heavy chain that can be used in a single-chain immunocytokine described herein can include a signal sequence having the amino acid sequence set forth in SEQ ID NO:6, a variable domain having the amino acid sequence set forth in SEQ ID NO:4, and a constant domain having the amino acid sequence set forth in SEQ ID NO:5. For example, an immunoglobulin heavy chain that can be used in a single-chain immunocytokine described herein can include the amino acid sequence set forth in SEQ ID NO:1. In some cases, an immunoglobulin heavy chain can have one or more modifications to the amino acid sequence (e.g., one or more modifications to SEQ ID NO:1). In some cases, a modification to the amino acid sequence of a heavy chain included in a single-chain immunocytokine described herein can alter the cytokine affinity of the single-chain immunocytokine. In some cases, a modification to the amino acid sequence of a heavy chain included in a single-chain immunocytokine described herein can alter the receptor competition of the single-chain immunocytokine.

A single-chain immunocytokine described herein (e.g., a single-chain immunocytokine that can bind to an IL-2Rα/IL-2Rβ/γc polypeptide complex) can include any appropriate IL-2 polypeptide (or fragment thereof) that can bind an IL-2Rα/IL-2Rβ/γc polypeptide complex. An IL-2 polypeptide (or fragment thereof) that can bind an IL-2Rα/IL-2Rβ/γc polypeptide complex can be from any source. In some cases, an IL-2 polypeptide (or fragment thereof) that can bind an IL-2Rα/IL-2Rβ/γc polypeptide complex can be a naturally occurring IL-2 polypeptide (or fragment thereof) that can bind an IL-2Rα/IL-2Rβ/γc polypeptide complex. In some cases, an

IL-2 polypeptide (or fragment thereof) that can bind an IL-2Rα/IL-2Rβ/γc polypeptide complex can be synthetic. An IL-2 polypeptide (or fragment thereof) that can bind an IL-2Rα/IL-2Rβ/γc polypeptide complex can have any appropriate sequence. In some cases, an IL-2 polypeptide (or fragment thereof) that can bind an IL-2Rα/IL-2Rβ/γc polypeptide complex can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:9. For example, a single-chain immunocytokine described herein can include an IL-2 polypeptide (or fragment thereof) that can bind an IL-2Rα/IL-2Rβ/γc polypeptide complex having the amino acid sequence set forth in SEQ ID NO:9. In some cases, an IL-2 polypeptide (or fragment thereof) that can bind an IL-2Rα/IL-2Rβ/γc polypeptide complex can have one or more modifications to the amino acid sequence (e.g., one or more modifications to SEQ ID NO:9). In some cases, a modification to the amino acid sequence of IL-2 polypeptide (or fragment thereof) that can bind an IL-2Rα/IL 2Rβ/γc polypeptide complex included in a single-chain immunocytokine described herein can mitigate disruption of the intramolecular assembly of the single-chain immunocytokine. In some cases, a modification to the amino acid sequence of a heavy chain included in a single-chain immunocytokine described herein can enhance the activity (e.g., signaling activity) of the single-chain immunocytokine.

A single-chain immunocytokine described herein (e.g., a single-chain immunocytokine that can bind to an IL-2Rα/IL-2Rβ/γc polypeptide complex) can include any appropriate immunoglobulin light chain. An immunoglobulin light chain can be from any appropriate type of immunoglobulin light chain (e.g., a (κ) light chain and a lambda (λ) light chain). In some cases, an immunoglobulin light chain can be a λ, light chain (e.g., a human λ light chain). An immunoglobulin light chain can have any appropriate light chain variable domain (V_{L}). An immunoglobulin light chain can have any appropriate light chain constant domain (C_{L}). An immunoglobulin light chain can be from any appropriate immunoglobulin. In some cases, the immunoglobulin light chain variable domain and the immunoglobulin light chain constant domains can be from the same immunoglobulin. In some cases, the immunoglobulin light chain variable domain and the immunoglobulin light chain constant domains can be from different immunoglobulins. In some cases, the immunoglobulin light chain variable domain and/or the immunoglobulin light chain constant domains can be from a naturally occurring immunoglobulin (e.g., can be derived from a naturally occurring immunoglobulin). In some cases, the immunoglobulin light chain variable domain and/or the immunoglobulin light chain constant domains can be synthetic. Examples of immunoglobulins whose light chain variable domain and/or the immunoglobulin light chain constant domains can be used in a single-chain immunocytokine described herein include, without limitation, monoclonal antibody F5111 light chains, monoclonal antibody F5111.4 light chains, monoclonal antibody F5111.7 light chains, monoclonal antibody F5111.8 light chains, and monoclonal antibody F5111.2 light chains. In some cases, immunoglobulins whose light chain variable domains and/or the light chain constant domains can be used in a single-chain immunocytokine described herein can be as described elsewhere (see, e.g., Trotta et al., Nat Med. 24(7):1005-1014 (2018)). An immunoglobulin light chain can include any appropriate sequence (e.g., amino acid sequence). In some cases, an immunoglobulin light chain variable domain can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:10. For example, a single-chain immunocytokine described herein can include an immunoglobulin light chain variable domain having the amino acid sequence set forth in SEQ ID NO:10. In some cases, an immunoglobulin light chain constant domain can include an amino acid sequence having at least about 70% identity (e.g., about 75%, about 80%, about 85%, about 88%, about 90%, about 93%, about 95%, about 97%, about, 98%, about 99%, or 100% sequence identity) to the amino acid sequence set forth in SEQ ID NO:11. For example, a single-chain immunocytokine described herein can include an immunoglobulin light chain constant domain having the amino acid sequence set forth in SEQ ID NO:11. In some cases, an immunoglobulin light chain also can include a signal sequence. A signal sequence can be any appropriate signal sequence (e.g., SEQ ID NO:7 and SEQ ID NO:8). For example, a single-chain immunocytokine described herein can include an immunoglobulin light chain having a signal sequence with the amino acid sequence set forth in SEQ ID NO:7.

An exemplary immunoglobulin light chain that can be used in a single-chain immunocytokine described herein (e.g., a single-chain immunocytokine that can bind to an IL-2Rα/IL-2Rβ/γc polypeptide complex) is set forth in SEQ ID NO:2. For example, an immunoglobulin light chain that can be used in a single-chain immunocytokine described herein can include a signal sequence, a variable domain from a F5111 antibody, and a λ constant domain (e.g., a human λ constant domain). For example, an immunoglobulin light chain that can be used in a single-chain immunocytokine described herein can include a signal sequence having the amino acid sequence set forth in SEQ ID NO:7, a variable domain having the amino acid sequence set forth in SEQ ID NO:10, and a constant domain having the amino acid sequence set forth in SEQ ID NO:11. In some cases, an immunoglobulin light chain that can be used in a single-chain immunocytokine described herein can include the amino acid sequence set forth in SEQ ID NO:2. In some cases, an immunoglobulin light chain can have one or more modifications to the amino acid sequence (e.g., one or more modifications to SEQ ID NO:2). In some cases, a modification to the amino acid sequence of a light chain included in a single-chain immunocytokine described herein can alter the cytokine affinity of the single-chain immunocytokine. In some cases, a modification to the amino acid sequence of a light chain included in a single-chain immunocytokine described herein can alter the receptor competition of the single-chain immunocytokine.

In some cases, an immunoglobulin light chain can include an IL-2 polypeptide (or fragment thereof) that can bind an IL-2Rα/IL-2R⊕/γc polypeptide complex described herein. In cases where an immunoglobulin light chain includes the IL-2 polypeptide (or fragment thereof) that can bind an IL-2Rα/IL-2Rβ/γc polypeptide complex, the IL-2 polypeptide (or fragment thereof) that can bind IL-2Rα/IL-2Rβ/γc polypeptide complex can be in any appropriate location within the immunoglobulin light chain. In some cases, the IL-2 polypeptide (or fragment thereof) that can bind an IL-2Rα/IL 2Rβ/γc polypeptide complex can be fused to the immunoglobulin light chain (e.g., the immunoglobulin light chain variable domain). When the IL-2 polypeptide (or fragment thereof) that can bind an IL-2Rα/IL-2Rβ/γc polypeptide complex and the immunoglobulin light chain variable domain are a fusion polypeptide, the IL-2 polypeptide (or fragment thereof) that can bind an IL-2Rα/IL-2Rβ/γc polypeptide complex and the immunoglobulin light chain variable domain can be fused via a linker. A linker can be any appropriate linker. In some cases, a linker can be flexible (e.g., to allow for intramolecular interaction(s)). In some cases, a linker can be a peptide linker. A peptide linker can include any appropriate number of amino acids. For example, a peptide linker can include from about 10 amino acids to about 60 amino acids (e.g., from about 10 amino acids to about 50 amino acids, from about 10 amino acids to about 40 amino acids, from about 10 amino acids to about 30 amino acids, from about 20 amino acids to about 60 amino acids, from about 30 amino acids to about 60 amino acids, from about 40 amino acids to about 60 amino acids, from about 50 amino acids to about 60 amino acids, from about 15 amino acids to about 55 amino acids, from about 20 amino acids to about 50 amino acids, from about 30 amino acids to about 40 amino acids, from about 20 amino acids to about 40 amino acids, from about 30 amino acids to about 50 amino acids, or from about 40 amino acids to about 60 amino acids). A peptide linker can include any appropriate amino acids. For example, a peptide linker can include one or more glycine (Gly) residues and/or one or more serine (Ser) residues. Examples of linkers that can be used to fuse an IL-2 polypeptide (or fragment thereof) that can bind an IL-2Rα/IL-2Rβ/γc polypeptide complex to an immunoglobulin light chain variable domain include, without limitation, a (Gly₄Ser)₂ linker (SEQ ID NO:12), a (Gly₄Ser)₃ linker (SEQ ID NO:13), a (Gly₄Ser)₄ linker (SEQ ID NO:14), a (Gly₄Ser)₅ linker (SEQ ID NO:15), a (Gly₄Ser)₆ linker (SEQ ID NO:16), a (Gly₄Ser)₇ linker (SEQ ID NO:17), a (Gly₄Ser)₈ linker (SEQ ID NO:18), a (Gly₄Ser)₉ linker (SEQ ID NO:19), a (Gly₄Ser)₁₀ linker (SEQ ID NO:20), a (Gly4Ser)₁₁ linker (SEQ ID NO:21), and a (Gly₄Ser)₁₂ linker (SEQ ID NO:22). For example, a single-chain immunocytokine described herein can include an immunoglobulin light chain having an IL-2 polypeptide (or fragment thereof) that can bind an IL-2Rα/IL-2β/γc polypeptide complex fused to an immunoglobulin light chain variable domain via a linker having the amino acid sequence set forth in SEQ ID NO:12, SEQ ID NO:13, SEQ ID NO:15, or SEQ ID NO:17. In some cases, an IL-2 polypeptide (or fragment thereof) that can bind an IL-2Rα/IL-2Rβ/γc polypeptide complex can have one or more modifications to the amino acid sequence (e.g., one or more modifications to SEQ ID NO:12, one or more modifications to SEQ ID NO:13, one or more modifications to SEQ ID NO:15, or one or more modifications to SEQ ID NO:17). In some cases, a modification to the amino acid sequence of a linker can alter the length, charge, structure, and/or composition of the linker.

Exemplary immunoglobulin light chains that include an IL-2 polypeptide (or fragment thereof) that can bind an IL-2Rα/IL 2Rβ/γc polypeptide complex that can be used in a single-chain immunocytokine described herein (e.g., a single-chain immunocytokine that can bind to an IL-2Rα/IL 2Rβ/γc polypeptide complex) are set forth in SEQ ID NO:3, SEQ ID NO:24, and SEQ ID NO:25. For example, an immunoglobulin light chain that can be used in a single-chain immunocytokine described herein can include a signal sequence, an IL-2 polypeptide (or fragment thereof) that can bind an IL-2Rα/IL 2Rβ/γc polypeptide complex, a linker, a variable domain from a F5111 antibody, and a λ constant domain (e.g., a human λ constant domain). For example, an immunoglobulin light chain that can be used in a single-chain immunocytokine described herein can include (a) a signal sequence having the amino acid sequence set forth in SEQ ID NO:8, (b) an IL-2 polypeptide having the amino acid sequence set forth in SEQ ID NO: 9, (c) a linker having the amino acid sequence set forth in SEQ ID NO:13, SEQ ID NO:15, or SEQ ID NO:17, (d) a variable domain having the amino acid sequence set forth in SEQ ID NO:10, and (e) a constant domain having the amino acid sequence set forth in SEQ ID NO:11. In some cases, an immunoglobulin light chain that can be used in a single-chain immunocytokine described herein can include the amino acid sequence set forth in SEQ ID NO:3, SEQ ID NO:24, or SEQ ID NO:25. In some cases, an immunoglobulin light chain can have one or more modifications to the amino acid sequence (e.g., one or more modifications to SEQ ID NO:3, one or more modifications to SEQ ID NO:24, or one or more modifications to SEQ ID NO:25). In some cases, a modification to the amino acid sequence of a light chain included in a single-chain immunocytokine described herein can alter the cytokine affinity of the single-chain immunocytokine. In some cases, a modification to the amino acid sequence of a light chain included in a single-chain immunocytokine described herein can alter the receptor competition of the single-chain immunocytokine.

In some cases, a single-chain immunocytokine described herein (e.g., a single-chain immunocytokine that can bind to an IL-2Rα/IL 2Rβ/γc polypeptide complex) can be a stable molecule (e.g., as compared to a molecule that can bind to an IL-2Rα/IL 2Rβ/γc polypeptide complex that is not present in a single-chain immunocytokine described herein). For example, a single-chain immunocytokine described herein can have a half-life (e.g., an in vivo half-life such as a serum half-life or a plasma half-life) of from about 5 minutes to about 6 months (e.g., from about 15 minutes to about 6 months, from about 30 minutes to about 6 months, from about 1 hour to about 6 months, from about 24 hours to about 6 months, from about 3 days to about 6 months, from about 7 days to about 6 months, from about 1 month to about 6 months, from about 3 months to about 6 months, from about 5 minutes to about 3 months, from about 5 minutes to about 1 month, from about 5 minutes to about 2 weeks, from about 5 minutes to about 7 days, from about 5 minutes to about 3 days, from about 5 minutes to about 24 hours, from about 5 minutes to about 12 hours, from about 5 minutes to about 60 minutes, from about 30 minutes to about 3 days, from about 3 days to about 1 week, from about 1 week to about 1 month, or from about 1 month to about 3 months). For example, a single-chain immunocytokine described herein can have a shelf life at standard room temperature conditions (e.g., about 25° C.) for from about 1 day to about 1 month (e.g., from about 1 day to about 2 weeks, from about 1 day to about 1 week, from about 1 day to about 5 days, from about 4 days to about 1 month, from about 1 week to about 1 month, from about 2 weeks to about 1 month, from about 3 days to about 2 weeks, from about 2 days to about 5 days, from about 5 days to about 2 weeks, or from about 1 week to about 3 weeks). Any appropriate method can be used to determine the stability of a single-chain immunocytokine described herein. For example, thermal shift assay, protein stability curve analysis, size exclusion chromatography, and/or dynamic light scattering can be used to determine the stability of a single-chain immunocytokine described herein.

In some cases, a single-chain immunocytokine described herein (e.g., a single-chain immunocytokine that can bind to an IL-2Rα/IL-2Rβ/γc polypeptide complex) can have an enhanced interaction with (e.g., stronger binding affinity for) an IL-2Ra polypeptide (e.g., as compared to a molecule that can bind to an IL-2Ra polypeptide that is not present in a single-chain immunocytokine described herein). For example, a single-chain immunocytokine described herein can have an affinity for an IL-2Rα/IL-2Rβ/γc polypeptide complex of from about 10 nM K_{D} to about 1 pM K_{D}.

In some cases, a single-chain immunocytokine described herein (e.g., a single-chain immunocytokine that can bind to an IL-2Rα/IL-2Rβ/γc polypeptide complex) can have a reduced or eliminated interaction with (e.g., weaker binding affinity for) an IL-2Rβ polypeptide (e.g., as compared to a molecule that can bind to an IL-2Rβ polypeptide that is not present in a single-chain immunocytokine described herein). For example, a single-chain immunocytokine described herein can have an affinity for an IL-2Rβ polypeptide of greater than about 300 nM K_{D}. Any appropriate method can be used to determine the binding affinity between a single-chain immunocytokine described herein (e.g., a single-chain immunocytokine that can bind to an IL-2Rα/IL-2Rβ/γc polypeptide complex) and an IL-2Rβ polypeptide and/or an IL2Rα polypeptide. For example, affinity titration studies, surface plasmon resonance, isothermal calorimetry, and/or bio-layer interferometry can be used to determine the binding affinity between a single-chain immunocytokine described herein and an IL-2Rβ polypeptide and/or an

IL-2Rα polypeptide.

In some cases, a single-chain immunocytokine described herein (e.g., a single-chain immunocytokine that can bind to an IL-2Rα/IL-2Rβ/γc polypeptide complex) can activate a reduced or eliminated number of Effs (e.g., as compared to a molecule that can bind to an IL-2Rα/IL-2Rβ/γc polypeptide complex that is not present in a single-chain immunocytokine described herein). For example, a single-chain immunocytokine described herein does not substantially activate Effs (e.g., does not active Effs to a detectable level and/or a level sufficient to induce an immune response). Any appropriate method can be used to determine the presence, absence, or amount of Effs. For example, immunostaining for Eff markers (e.g., CD4, CD8, CD16, CD56, NK1.1, NK1.2, CD44, and/or CD62L) can be used to determine the presence, absence, or amount of Effs.

This document also provides methods and materials for making single-chain immunocytokines described herein (e.g., a single-chain immunocytokine that can bind to an IL-2Rα/IL 2Rβ/γc polypeptide complex). For example, this document also provides nucleic acid (e.g., nucleic acid vectors) that can encode a polypeptide that can be used to generate single-chain immunocytokines described herein are provided. In some cases, nucleic acid can encode an immunoglobulin heavy chain, an IL-2 polypeptide (or fragment thereof) that can bind an IL-2Rα/IL 2Rβ/γc polypeptide complex, and/or an immunoglobulin light chain that can be used to generate a single-chain immunocytokine that can bind to an IL-2Rα/IL 2Rβ/γc polypeptide complex. For example, a first nucleic acid can encode an immunoglobulin heavy chain, and a second nucleic acid can encode an IL-2 polypeptide (or fragment thereof) that can bind an IL-2Rα/IL 2Rβ/γc polypeptide complex fused to an immunoglobulin light chain.

Nucleic acid (e.g., nucleic acid vectors) encoding one or more polypeptides (e.g., an immunoglobulin heavy chain, an IL-2 polypeptide (or fragment thereof) that can bind an IL-2Rα/IL 2Rβ/γc polypeptide complex, and/or an immunoglobulin light chain) that can be used to generate polypeptide that can be used to generate single-chain immunocytokines described herein (e.g., a single-chain immunocytokine that can bind to an IL-2Rα/IL 2Rβ/γc polypeptide complex) can be any appropriate nucleic acid. Nucleic acid can be DNA (e.g., a DNA construct), RNA (e.g., mRNA), or a combination thereof. In some cases, nucleic acid encoding one or more polypeptides that can be used to generate polypeptide that can be used to generate single-chain immunocytokines described herein can be a vector (e.g., an expression vector or a plasmid).

In some cases, nucleic acid encoding one or more polypeptides (e.g., an immunoglobulin heavy chain, an IL-2 polypeptide (or fragment thereof) that can bind an IL-2Rα/IL 2Rβ/γc polypeptide complex, and/or an immunoglobulin light chain) that can be used to generate polypeptide that can be used to generate single-chain immunocytokines described herein (e.g., a single-chain immunocytokine that can bind to an IL-2Rα/IL-2Rβ/γc polypeptide complex) also can include one or more regulatory elements (e.g., to regulate expression of the amino acid chain). Examples of regulatory elements that can be included in nucleic acid encoding one or more polypeptides that can be used to generate polypeptide that can be used to generate single-chain immunocytokines described herein include, without limitation, promoters (e.g., constitutive promoters, tissue/cell-specific promoters, and inducible promoters such as chemically-activated promoters and light-activated promoters), and enhancers.

In some cases, one or more polypeptides (e.g., an immunoglobulin heavy chain, an IL-2 polypeptide (or fragment thereof) that can bind an IL-2Rα/IL-2Rβ/γc polypeptide complex, and/or an immunoglobulin light chain) encoded by nucleic acid described herein can be used to generate single-chain immunocytokines described herein (e.g., a single-chain immunocytokine that can bind to an IL-2Rα/IL-2Rβ/γc polypeptide complex). For example, two or more polypeptides including an immunoglobulin heavy chain, an IL-2 polypeptide (or fragment thereof) that can bind an IL-2Rα/IL-2Rβ/γc polypeptide complex, and an immunoglobulin light chain can assemble (e.g., can self-assemble) into a single-chain immunocytokine described herein (e.g., a single-chain immunocytokine that can bind to an IL-2Rα/IL 2Rβ/γc polypeptide complex). In some cases, an immunoglobulin heavy chain encoded by a first nucleic acid, and an IL-2 polypeptide (or fragment thereof) that can bind an IL-2Rα/IL-2Rβ/γc polypeptide complex fused to an immunoglobulin light chain encoded by a second nucleic acid can assemble (e.g., can self-assemble) into a single-chain immunocytokine described herein. When two or more polypeptides including an immunoglobulin heavy chain, an IL-2 polypeptide (or fragment thereof) that can bind an IL-2Rα/IL-2Rβ/γc polypeptide complex, and an immunoglobulin light chain assemble into a single-chain immunocytokine described herein, the two or more polypeptides can assemble in vivo or in vitro.

In some cases, single-chain immunocytokines described herein (e.g., a single-chain immunocytokine that can bind to an IL-2Rα/IL-2Rβ/γc polypeptide complex), or nucleic acid encoding one or more polypeptides (e.g., an immunoglobulin heavy chain, an IL-2 polypeptide (or fragment thereof) that can bind an IL-2Rα/IL-2Rβ/γc polypeptide complex, and/or an immunoglobulin light chain) that can be used to generate polypeptide that can be used to generate single-chain immunocytokines described herein, can be purified. A “purified” polypeptide or nucleic acid refers to a polypeptide or nucleic acid that constitutes the major component in a mixture of components, e.g., 30% or more, 40% or more, 50% or more, 60% or more, 70% or more, 80% or more, 90% or more, 95% or more, or 99% or more by weight. For example, a purified single-chain immunocytokine can constitute about 30% or more by weight of a composition containing one or more single-chain immunocytokines. Polypeptides may be purified by methods including, but not limited to, affinity chromatography and immunosorbent affinity column. For example, a purified nucleic acid encoding one or more polypeptides that can be used to generate single-chain immunocytokines described herein can constitute about 30% or more by weight of a composition containing one or more amino acid chains that can be used to generate a single-chain immunocytokine described herein. Nucleic acid may be purified by methods including, but not limited to, phenol-chloroform extraction and column purification (e.g., mini-column purification).

Also provided herein are methods and materials for treating a mammal (e.g., a human) in need thereof (e.g., a mammal having a condition that can benefit from reducing or eliminating an immune response within the mammal such as an autoimmune disease and/or transplant rejection). In some cases, a composition containing one or more single-chain immunocytokines described herein (e.g., a single-chain immunocytokine that can bind to an IL-2Rα/IL-2Rβ/γc polypeptide complex), or nucleic acid encoding one or more polypeptides (e.g., an immunoglobulin heavy chain, an IL-2 polypeptide (or fragment thereof) that can bind an IL-2Rα/IL 2Rβ/γc polypeptide complex, and/or an immunoglobulin light chain) that can be used to a generate single-chain immunocytokine described herein, can be used for treating a mammal having an autoimmune disease. For example, a composition containing one or more single-chain immunocytokines described herein, or nucleic acid encoding one or more polypeptides that can be used to generate single-chain immunocytokines described herein, can be administered a mammal having an autoimmune disease to treat the mammal. In some cases, a composition containing one or more single-chain immunocytokines described herein (e.g., a single-chain immunocytokine that can bind to an IL-2Rα/IL-2Rβ/γc polypeptide complex), or nucleic acid encoding one or more polypeptides (e.g., an immunoglobulin heavy chain, an IL-2 polypeptide (or fragment thereof) that can bind an IL-2Rα/IL-2Rβ/γc polypeptide complex, and/or an immunoglobulin light chain) that can be used to generate a single-chain immunocytokine described herein, can be used for treating a mammal having transplant rejection. For example, a composition containing one or more single-chain immunocytokines described herein, or nucleic acid encoding one or more polypeptides that can be used to generate single-chain immunocytokines described herein, can be administered a mammal having transplant rejection to treat the mammal.

Any appropriate mammal having an autoimmune disease can be treated as described herein (e.g., by administering a composition containing one or more single-chain immunocytokines that can bind to an IL-2Rα/IL-2Rβ/γc polypeptide complex). Examples of mammals that can be treated as described herein include, without limitation, primates (e.g., humans and monkeys), dogs, cats, horses, cows, pigs, sheep, rabbits, mice, and rats. For example, humans having an autoimmune disease can be treated with a composition containing one or more single-chain immunocytokines that can bind to an IL-2Rα/IL-2Rβ/γc polypeptide complex. When treating a mammal having an autoimmune disease as described herein (e.g., by administering a composition containing one or more single-chain immunocytokines that can bind to an IL-2Rα/IL-2Rβ/γc polypeptide complex), the mammal can have any type of autoimmune disease. Examples of autoimmune diseases that can be treated as described herein include, without limitation, type 1 diabetes, multiple sclerosis, Chron's disease, ulcerative colitis, psoriasis, graft-versus-host disease, Guillain-Barre syndrome, lupus, rheumatoid arthritis, chronic inflammatory demyelinating polyneuropathy, Hashimoto Thyroiditis, Celiac disease, Addison disease, autoimmune hepatitis, antiphospholipid syndrome, and Graves disease.

Any appropriate method can be used to identify a mammal (e.g., a human) as having an autoimmune disease. For example, laboratory tests (e.g., antinuclear antibody test (ANA)), symptom analysis, physical examination, MRI, and/or CT scan can be used to identify mammals (e.g., humans) having an autoimmune disease.

When treating a mammal having, or at risk of developing, transplant rejection as described herein (e.g., by administering a composition containing one or more single-chain immunocytokines that can bind to an IL-2Rα/IL-2Rβ/γc polypeptide complex), the mammal can have, or can be preparing to have, a transplant of any appropriate organ and/or tissue. Examples of organs and tissues that can be transplanted in a mammal that can be treated as described herein include, without limitation, skin, bone, blood, heart, liver, kidney, pancreas, intestine, stomach, testis, penis, cornea, bone marrow, and lung. A transplant can be an allogeneic transplant or an autologous transplant. In some cases, the materials and methods described herein also can be used to treat a mammal having a complication or disease associated with a transplant (e.g., a graft versus host disease).

Any appropriate method can be used to identify a mammal (e.g., a human) as having transplant rejection. For example, laboratory tests (e.g., ANA), symptom analysis, physical examination, organ biopsy, and/or CT scan can be used to identify mammals (e.g., humans) having transplant rejection.

Once identified as having an autoimmune disease and/or as having, or as being at risk of developing, transplant rejection, a mammal (e.g., a human) can be administered, or instructed to self-administer, a composition containing one or more single-chain immunocytokines described herein (e.g., a single-chain immunocytokine that can bind to an IL-2Rα/IL-2Rβ/γc polypeptide complex). In some cases, a composition containing one or more single-chain immunocytokines described herein can be used to reduce the number of autoantibodies present in a mammal (e.g., a mammal having an autoimmune disease and/or having transplant rejection). In some cases, a composition containing one or more single-chain immunocytokines described herein can be used to reduce or eliminate one or more symptoms within a mammal having an autoimmune disease. In some cases, one or more single-chain immunocytokines described herein (e.g., a single-chain immunocytokine that can bind to an IL-2Rα/IL-2Rβ/γc polypeptide complex) can be administered to a mammal having an autoimmune disease as the sole active ingredients used to treat an autoimmune disease and/or a transplant rejection.

In some cases, where one or more single-chain immunocytokines described herein (e.g., a single-chain immunocytokine that can bind to an IL-2Rα/IL-2Rβ/γc polypeptide complex) are administered to a mammal having an autoimmune disease, the one or more single-chain immunocytokines described herein can be administered as a combination therapy with one or more additional treatments used to treat an autoimmune disease and/or one or more additional immunosuppressants. For example, a combination therapy used to treat an autoimmune disease can include administering to the mammal (e.g., a human) one or more single-chain immunocytokines described herein and one or more autoimmune disease treatments such as an adoptive cell (e.g., T_{Reg}) transfer, tolerogenic vaccination, an immune checkpoint agonist, and/or steroid administration. For example, a combination therapy used to enhance an immune response can include administering to the mammal (e.g., a human) one or more single-chain immunocytokines described herein and one or more immunosuppressants such as cyclosporine, rapamycin, methotrexate, azathioprine, chlorambucil, leflunomide, and/or mycophenolate mofetil.

In some cases, where one or more single-chain immunocytokines described herein (e.g., a single-chain immunocytokine that can bind to an IL-2Rα/IL-2Rβ/γc polypeptide complex) are administered to a mammal having, or at risk of developing, transplant rejection, the one or more single-chain immunocytokines described herein can be administered as a combination therapy with one or more additional treatments used to treat transplant rejection. For example, a combination therapy used to treat transplant rejection can include administering to the mammal (e.g., a human) one or more single-chain immunocytokines described herein and one or more additional immunosuppressants such as cyclosporine, rapamycin, methotrexate, azathioprine, chlorambucil, leflunomide, and/or mycophenolate mofetil.

In cases where one or more single-chain immunocytokines described herein are used in combination with one or more additional treatments, the one or more additional treatments can be administered at the same time or independently. For example, one or more single-chain immunocytokines described herein can be administered first, and the one or more additional treatments can be administered second, or vice versa.

In some cases, one or more single-chain immunocytokines described herein (e.g., a single-chain immunocytokine that can bind to an IL-2Rα/IL-2Rβ/γc polypeptide complex) can be formulated into a composition (e.g., pharmaceutically acceptable composition) for administration to a mammal in need thereof (e.g., a mammal having a condition that can benefit from reducing or eliminating an immune response within the mammal such as an autoimmune disease and/or transplant rejection). For example, a therapeutically effective amount of one or more single-chain immunocytokines described herein can be formulated together with one or more pharmaceutically acceptable carriers (additives) and/or diluents. A pharmaceutical composition can be formulated for administration in any appropriate dosage form. Examples of dosage forms include solid or liquid forms including, without limitation, gums, capsules, tablets (e.g., chewable tablets, and enteric coated tablets), suppository, liquid, enemas, suspensions, solutions (e.g., sterile solutions), sustained-release formulations, delayed-release formulations, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol such as Vitamin E TPGS, sodium carboxymethylcellulose, polyacrylates, waxes, polyethylenepolyoxypropylene-block polymers, and wool fat.

A composition (e.g., a pharmaceutical composition) containing one or more single-chain immunocytokines described herein (e.g., a single-chain immunocytokine that can bind to an IL-2Rα/IL-2Rβ/γc polypeptide complex) can be designed for oral or parenteral (including subcutaneous, intratumoral, intramuscular, intravenous, and intradermal) administration. When being administered orally, a pharmaceutical composition containing one or more single-chain immunocytokines described herein can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more single-chain immunocytokines described herein (e.g., a single-chain immunocytokine that can bind to an IL-2Rα/IL-2Rβ/γc polypeptide complex) can be administered locally or systemically. For example, a composition containing one or more single-chain immunocytokines described herein can be administered systemically by an oral administration or by injection to a mammal (e.g., a human).

Effective doses of one or more single-chain immunocytokines described herein (e.g., a single-chain immunocytokine that can bind to an IL-2Rα/IL-2Rβ/γc polypeptide complex) can vary depending on the severity of the autoimmune disease, the route of administration, the age and general health condition of the subject, excipient usage, the possibility of co-usage with other therapeutic treatments such as use of other agents, and/or the judgment of the treating physician.

An effective amount of a composition containing one or more single-chain immunocytokines described herein (e.g., a single-chain immunocytokine that can bind to an

IL-2Rα/IL-2Rβ/γc polypeptide complex) can be any amount that can treat a mammal (e.g., a mammal having an autoimmune disease and/or having, or at risk of developing, transplant rejection) without producing significant toxicity to the mammal. An effective amount of a single-chain immunocytokine described herein can be any appropriate amount. In some cases, an effective amount of a single-chain immunocytokine described herein can be from about 0.05 milligrams (mg) to about 500 mg per kg of body weight (mg/kg; e.g., from about 0.05 mg/kg to about 400 mg/kg, from about 0.05 mg/kg to about 300 mg/kg, from about 0.05 mg/kg to about 200 mg/kg, from about 0.05 mg/kg to about 100 mg/kg, from about 0.05 mg/kg to about 50 mg/kg, from about 0.5 mg/kg to about 500 mg/kg, from about 1 mg/kg to about 500 mg/kg, from about 50 mg/kg to about 500 mg/kg, from about 100 mg/kg to about 500 mg/kg, from about 200 mg/kg to about 500 mg/kg, from about 300 mg/kg to about 500 mg/kg, from about 400 mg/kg to about 500 mg/kg, from about 0.5 mg/kg to about 400 mg/kg, from about 1 mg/kg to about 300 mg/kg, from about 50 mg/kg to about 200 mg/kg, from about 1 mg/kg to about 100 mg/kg, from about 100 mg/kg to about 200 mg/kg, from about 200 mg/kg to about 300 mg/kg, or from about 300 mg/kg to about 400 mg/kg body weight) of a mammal (e.g., a human). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the condition (e.g., a condition that can benefit from reducing or eliminating an immune response within the mammal such as an autoimmune disease and/or transplant rejection) may require an increase or decrease in the actual effective amount administered.

The frequency of administration of a composition containing one or more single-chain immunocytokines described herein (e.g., a single-chain immunocytokine that can bind to an IL-2Rα/IL-2Rβ/γc polypeptide complex) can be any frequency that can treat a mammal (e.g., a mammal having an autoimmune disease and/or having, or at risk of developing, transplant rejection) without producing significant toxicity to the mammal. For example, the frequency of administration can be from about three times a day to about once a week, from about twice a day to about twice a week, or from about once a day to about twice a week. The frequency of administration can remain constant or can be variable during the duration of treatment. A course of treatment with a composition containing one or more single-chain immunocytokines described herein can include rest periods. For example, a composition containing one or more single-chain immunocytokines described herein can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and/or severity of the condition (e.g., a condition that can benefit from reducing or eliminating an immune response within the mammal such as an autoimmune disease and/or transplant rejection) may require an increase or decrease in administration frequency.

An effective duration for administering a composition containing one or more single-chain immunocytokines described herein (e.g., a single-chain immunocytokine that can bind to an IL-2Rα/IL-2Rβ/γc complex) can be any duration that treat a mammal (e.g., a mammal having an autoimmune disease and/or having, or at risk of developing, transplant rejection) without producing significant toxicity to the mammal. For example, the effective duration can vary from several days to several weeks, months, or years. In some cases, the effective duration for the treatment of a mammal can range in duration from about one month to about 10 years. In some cases, the effective duration for the treatment of a mammal can be a chronic treatment (e.g., for the duration of the life of the mammal). Multiple factors can influence the actual effective duration used for a particular treatment. For example, an effective duration can vary with the frequency of administration, effective amount, use of multiple treatment agents, route of administration, and/or severity of the condition (e.g., a condition that can benefit from reducing or eliminating an immune response within the mammal such as an autoimmune disease and/or transplant rejection) being treated.

In some cases, the autoimmune disease present within a mammal, and/or the severity of one or more symptoms of the autoimmune disease being treated can be monitored. For example, the presence of autoantibodies present within a mammal being treated can be monitored. Any appropriate method can be used to determine whether or not the level of autoantibodies present within a mammal is reduced.

Alternatively, the methods and materials described herein can be used for treating a mammal (e.g., a human) having another condition that can benefit from reducing or eliminating an immune response within the mammal.

The invention will be further described in the following examples, which do not limit the scope of the invention described in the claims.

### EXAMPLES

Materials and Methods

Protein expression and purification

The published V_{H} and V_{L} sequences of F5111 (see, e.g., Trotta et al., Nat Med. 24(7):10051014 (2018)) were used to formulate the recombinant antibodies on the human immunoglobulin (IgG) 1 lambda isotype platform (SEQ ID NO:1 and SEQ ID NO:2). The heavy chain (HC) and light chain (LC) of the F5111 antibody were separately cloned into the gWiz vector (Genlantis). Antibodies were expressed recombinantly in human embryonic kidney (HEK) 293F cells via transient co-transfection of plasmids encoding the HC and LC. HC and LC plasmids were titrated in small-scale co-transfection tests to determine optimal ratios for large-scale expression. Secreted antibodies were purified from cell supernatants 5 days post-transfection via protein G affinity chromatography followed by size-exclusion chromatography on a Superdex 200 column (GE Healthcare) on an FPLC instrument, equilibrated in HEPES-buffered saline (HBS, 150 mM NaCl in 10 mM HEPES pH 7.3). Purity (>99%) was verified by SDS-PAGE analysis. For IC production, the hIL-2 cytokine was fused to the full F5111 antibody at the N-terminus of the LC, connected by either a flexible 15-amino acid (Gly₄Ser)₃ linker (F5111 IC LN15, SEQ ID NO:3), 25-amino acid (Gly₄Ser)₅ linker (F5111 IC LN25, SEQ ID NO:24), or a 35-amino acid (Gly₄Ser)₇ linker (F5111 IC LN35, SEQ ID NO:25) to allow for intramolecular interaction. Separate plasmids were prepared in the gWiz vector (Genlantis) encoding the F5111 HC and the hIL-2-fused F5111 LC. ICs were expressed and purified via transient co-transfection of HEK 293F cells, as described for the F5111 antibody.

The full hIL-2 cytokine (residues 1-133) and the extracellular domains of the hIL-2Rα (residues 1-214) and hIL-2R13 (residues 1-214) receptor subunits were cloned into the gWiz vector (Genlantis) with a C-terminal hexahistidine tag. Proteins were expressed via transient transfection of HEK 293F cells, as described for HEK, and purified via Ni-NTA affinity chromatography followed by followed by size-exclusion chromatography on a Superdex 200 column (GE Healthcare) on an FPLC instrument, equilibrated in HBS. Purity (>99%) was verified by SDS-PAGE analysis.

For expression of biotinylated hIL-2Ra and hIL-2Rβ, protein containing a C-terminal biotin acceptor peptide (BAP) (SEQ ID NO:23) was expressed and purified via Ni-NTA affinity chromatography and then biotinylated with the soluble BirA ligase enzyme in 0.5 mM Bicine pH 8.3, 100 mM ATP, 100 mM magnesium acetate, and 500 mM biotin (Sigma). Excess biotin was removed by size exclusion chromatography on a Superdex 200 column (GE Healthcare) on an FPLC instrument, equilibrated in HBS.

Cell Lines

HEK 293F cells were cultivated in Freestyle 293 Expression Medium (Thermo) supplemented with 0.01% penicillin-streptomycin (Gibco). Unmodified YT-1 and IL-2Rα YT-1 human natural killer cells (see, e.g., Kuziel et al., J Immunol. 150(8):3357-3365 (1993)) were cultured in RPMI complete medium (RPMI 1640 medium supplemented with 10% fetal bovine serum, 2 mM L-glutamine, minimum nonessential amino acids, sodium pyruvate, 25 mM HEPES, and penicillin-streptomycin [Gibco]) and maintained at 37° C. in a humidified atmosphere with 5% CO₂.

Yeast Surface Binding Studies

For antibody binding studies on yeast, hIL-2 (residues 1-133) or mouse IL-2 (mIL-2; residues 1-149) were cloned into the pCT302 vector and presented on the surface of yeast, as described elsewhere (see, e.g., Boder et al., Nat. Biotechnol., 15(6): 553-557 (1997)). Yeast displaying human or mouse IL-2 were incubated in PBSA containing serial dilutions of recombinant F5111 antibody ECD for 2 hours at room temperature. Cells were then washed and stained with a 1:200 dilution of AlexaFluor® 647-complexed streptavidin (Thermo) in PB SA for 15 minutes at 4° C. After a final wash, cells were analyzed for antibody binding using a CytoFLEX flow cytometer (Beckman Coulter). Background-subtracted and normalized binding curves were fitted to a first-order binding model and equilibrium dissociation constant (K_{D}) values were determined using GraphPad Prism. Studies were performed three times with similar results.

Bio-Layer Interferometry Binding Studies

For IL-2 versus immunocytokine affinity titration studies, biotinylated human IL-2 cytokine or IL-2Rα or IL-2Rβ receptor chains were immobilized to streptavidin-coated tips for analysis on an OCTET® Red96 bio-layer interferometry (BLI) instrument (ForteBio). Less than 5 signal units (nm) of receptor was immobilized to minimize mass transfer effects. Tips were exposed to serial dilutions of hIL-2, IL-2/F5111 complex, or single-chain IL-2/F5111 IC constructs in a 96-well plate for 300 seconds and dissociation was measured for 600 seconds. An irrelevant protein (the human monoclonal antibody trastuzumab) was included in a reference well to subtract non-specific binding. Surface regeneration for all interactions was conducted using a 15 second exposure to 0.1 M glycine pH 3.0. Experiments were carried out in PB SA (phosphate-buffered saline, pH 7.3 plus 0.1% bovine serum albumin (BSA, Thermo Fisher Scientific)) at 25° C. Data was visualized and processed using the Octet® Data Analysis software version 7.1 (Molecular Devices). Equilibrium titration curve fitting and K_{D} value determination was implemented using GraphPad Prism, assuming all binding interactions to be first order. Experiments were reproduced two times with similar results.

YT-1 Cell STATS Phosphorylation Studies

Approximately 2×10⁵ IL-2Rα⁻YT-1 or IL-2Rα⁺YT-1 cells were plated in each well of a 96-well plate and resuspended in RPMI complete medium containing serial dilutions of the indicated treatments. Cytokine/antibody complexes were formed by incubating a 1:1 molar ratio of F5111 antibody to hIL-2 for 1 hour at 37° C. Cells were stimulated for 20 minutes at 37° C. and immediately fixed by addition of formaldehyde to 1.5% and 10 minutes incubation at room temperature. Permeabilization of cells was achieved by resuspension in ice-cold 100% methanol for 30 minutes at 4° C. Fixed and permeabilized cells were washed twice with FACS buffer (phosphate-buffered saline [PBS] pH 7.2 containing 0.1% BSA [Thermo Fisher Scientific]) and incubated with Alexa Fluor® 647-complexed anti-STATS pY694 (BD Biosciences) diluted in FACS buffer for 2 hours at room temperature. Cells were then washed twice in FACS buffer and MFI was determined on a CytoFLEX flow cytometer (Beckman-Coulter). Dose-response curves were fitted to a logistic model and half-maximal effective concentrations (EC50s) were calculated using GraphPad Prism data analysis software after subtraction of the mean fluorescence intensity (MFI) of unstimulated cells and normalization to the maximum signal intensity. Experiments were conducted in triplicate and performed three times with similar results.

Human PBMC STATS Phosphorylation Studies

Human PBMCs were isolated from whole blood of healthy donors via Ficoll gradient following manufacturer protocols and then incubated with ACK lysis buffer for removal of red blood cells. Approximately 1×10⁶ human PBMCs were plated in each well of a 96-well plate and re-suspended in RPMI complete medium containing serial dilutions of the indicated treatments. Cytokine/antibody complexes were formed by incubating a 1:1 molar ratio of F5111 antibody to hIL-2 for 1 hour at 37° C. Cells were stimulated for 20 minutes at 37° C. and immediately fixed by addition of 1X Fix/Perm Buffer (BD Biosciences) and 50 minute incubation at 4° C. Permeabilization of cells was achieved by resuspension in Perm Buffer III (BD Biosciences) overnight at −20° C. Fixed and permeabilized cells were washed twice with FACS buffer (phosphate buffered saline [PBS] pH 7.2 containing 0.1% BSA [Thermo Fisher Scientific]) and incubated with an appropriate panel of anti-human antibodies (for human PBMCs: anti-CD3, anti-CD4, anti-CD8, anti-FOXP3, anti-CD25, anti-CD127, and anti-phosphorylated STATS) diluted in FACS buffer for 2 hours at room temperature. Cells were then washed twice in FACS buffer and MFI was determined on a CytoFLEX flow cytometer (Beckman-Coulter). Dose-response curves were fitted to a logistic model and half-maximal effective concentrations (EC50s) were calculated using GraphPad Prism data analysis software after subtraction of the mean fluorescence intensity (MFI) of unstimulated cells and normalization to the maximum signal intensity. Experiments were conducted in triplicate and performed two times with similar results.

### EXAMPLE 1

Engineered cytokine/antibody fusion for targeted expansion of human regulatory T cells

Administration of human IL-2 (hIL-2) in complex with the F5111 antibody was found to expand T_{RegS} but not effector T cells from human peripheral blood and in humanized mouse models, presenting an enticing opportunity for targeted cytokine therapy. It was further shown that IL-2/F5111 complex treatment reduces T1D severity in mice (Trotta et al., Nat Med. 24(7):1005-1014 (2018)). This exciting targeted IL-2 therapy holds vast clinical potential, but therapeutic development of a mixed cytokine/antibody complex is limited by dosing ratio considerations and complex instability. In fact, dissociation of the complex could induce dangerous toxicities from the free cytokine and potentially even exacerbate autoimmune pathogenesis by activating autoreactive effector T cells. Moreover, the free cytokine clears in <5 minutes from the bloodstream (Donohue et al., J Immunol. 130(5):2203-2208 (1983)).

This Example describes the design and engineering of a clinically relevant single-chain fusion protein (termed an immunocytokine, IC) that specifically stimulates T_{RegS}to combat pathogenic autoimmunity. The IC comprises the F5111 antibody and IL-2 in mammalian cells.

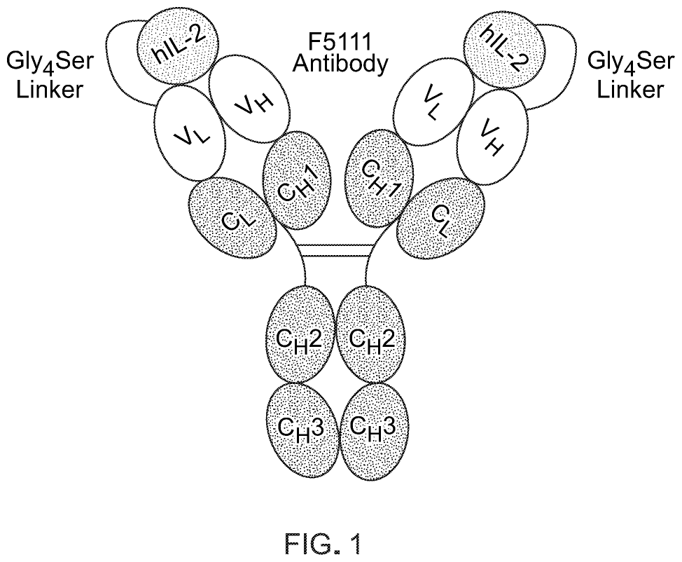

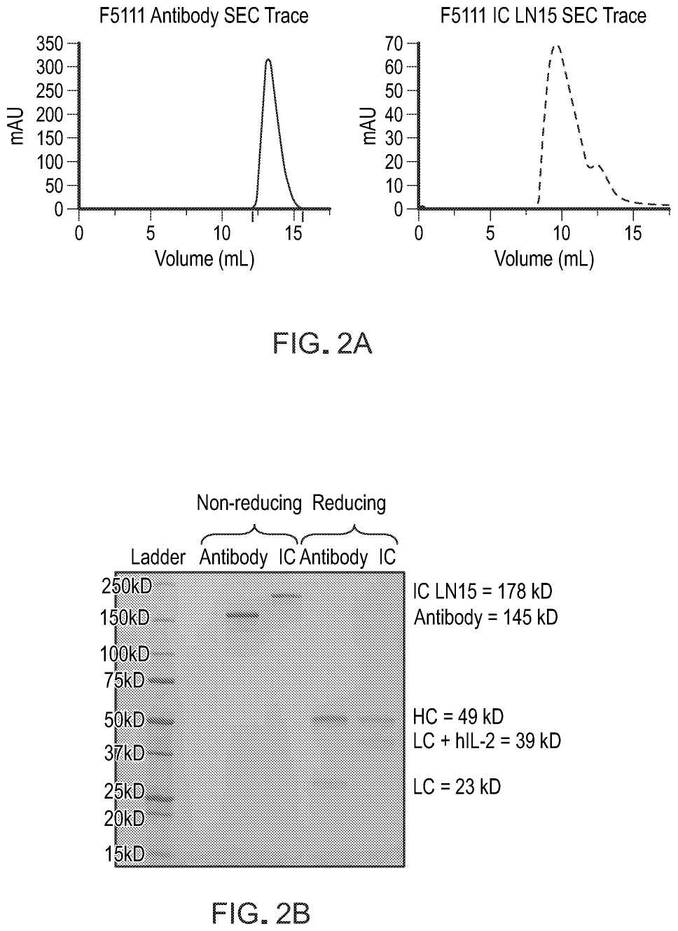

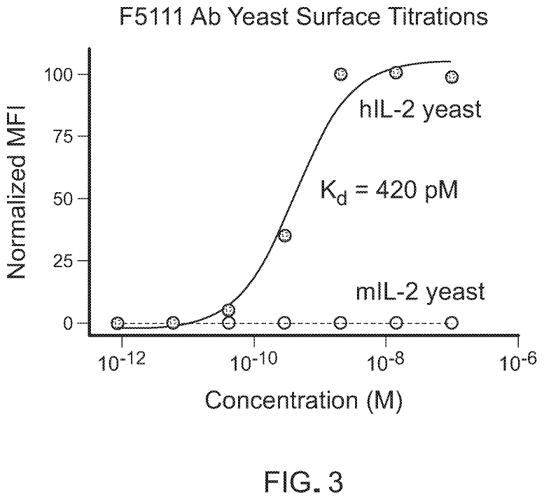

To combine the potency of cytokines with the pharmaceutically favorable properties of antibodies in a unimolecular targeted construct, human IL-2 (hIL-2) was fused to the cytokine-biasing F5111 antibody to create an immunocytokine (IC) (FIG. 1). The full hIL-2 cytokine was fused to the full-length F5111 human IgG1 lambda antibody at the LC N-terminus, connected by a flexible 15-amino acid (Gly₄Ser)₃ linker. This will be referred to as F5111 IC LN15. A rapid small-scale HEK 293F cell transfection assay was used to optimize immunocytokine expression. Cells were transfected in 6-well plates with predefined ratios of heavy chain (HC) and IL-2-fused light chain (LC) plasmid DNA. After a 3-day incubation, secreted protein was captured from the supernatant with protein G resin, eluted with 0.1 M glycine pH 2.0, and analyzed via SDS-PAGE. Titration of the HC:LC ratio revealed the optimal expression conditions. Immunocytokine expression was scaled up in HEK 293F cells and the secreted protein was purified via protein G affinity chromatography followed by size-exclusion chromatography. The process described above was also performed for expression of the recombinant F5111 antibody. F5111 antibody and F5111 IC LN15 were purified to homogeneity on an FPLC instrument (FIG. 2A), and purity (>99%) was verified via SDS-PAGE analysis (FIG. 2B). To verify binding of the recombinant F5111 antibody to the target hIL-2 cytokine, soluble antibody was titrated binding to yeast-displayed hIL-2 or mIL-2. As expected, the antibody bound hIL-2 with an apparent bivalent affinity of K_{D}=420 pM. The antibody did not cross react with mIL-2 (FIG. 3).

### EXAMPLE 2

This example demonstrates that the recombinantly expressed single-chain F5111 IC is properly assembled and does not bind to IL-2Rβ.

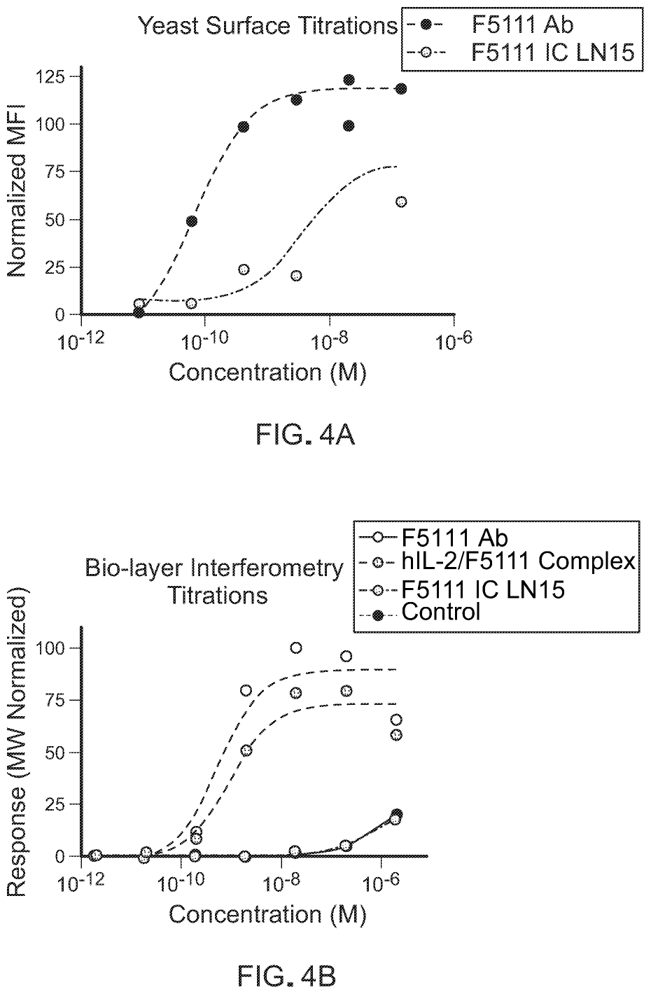

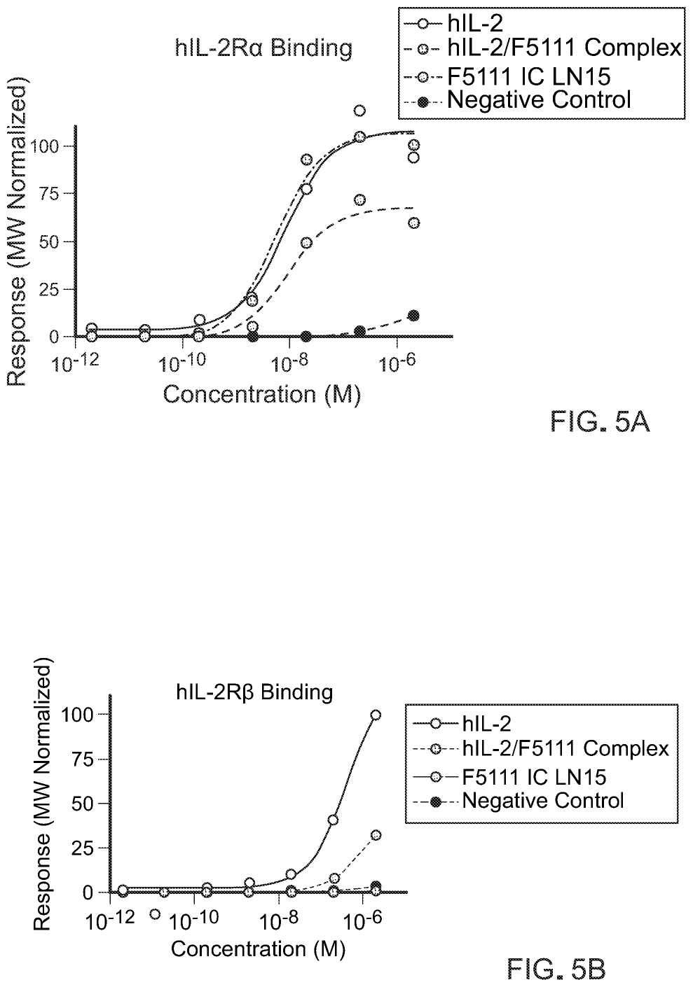

To demonstrate that hIL-2 is intramolecularly bound to the F5111 antibody within the IC, the binding affinity of F5111 IC LN15 for hIL-2 was measured and compared to that of recombinant F5111 antibody (Ab). The binding of purified F5111 antibody and IC to yeast surface-displayed hIL-2, as measured by flow cytometry, is shown in FIG. 4A. The binding affinities were also evaluated using bio-layer interferometry on an OCTET® instrument with biotinylated hIL-2 immobilized on a streptavidin-coated tip (FIG. 4B). For both yeast surface and bio-layer interferometry studies, a significant reduction in binding affinity was observed for F5111 IC LN15 relative to F5111 Ab, confirming intramolecular cytokine/antibody assembly. Bio-layer interferometry based binding studies were also conducted to assess the interaction between F5111 IC LN15 and the IL-2Rα and IL-2Rβ receptor chains, to ensure that the single-chain antibody/cytokine fusion was biased toward engagement of IL-2Rα (which is highly expressed on T_{Reg} cells but not effector cells) compared with IL-2Rβ. Indeed, biophysical assessment showed that F5111 IC LN15 bound IL-2Rα with equal potency to the free IL-2 cytokine (FIG. 5A), whereas F5111 IC LN15 exhibited significantly impaired binding to IL-2Rβ relative to the free IL-2 cytokine (FIG. 5B). These data corroborate the proper folding, intramolecular binding, and activity of the IC.

### EXAMPLE 3

This example demonstrates that the immunocytokine selectively biases IL-2 signaling.

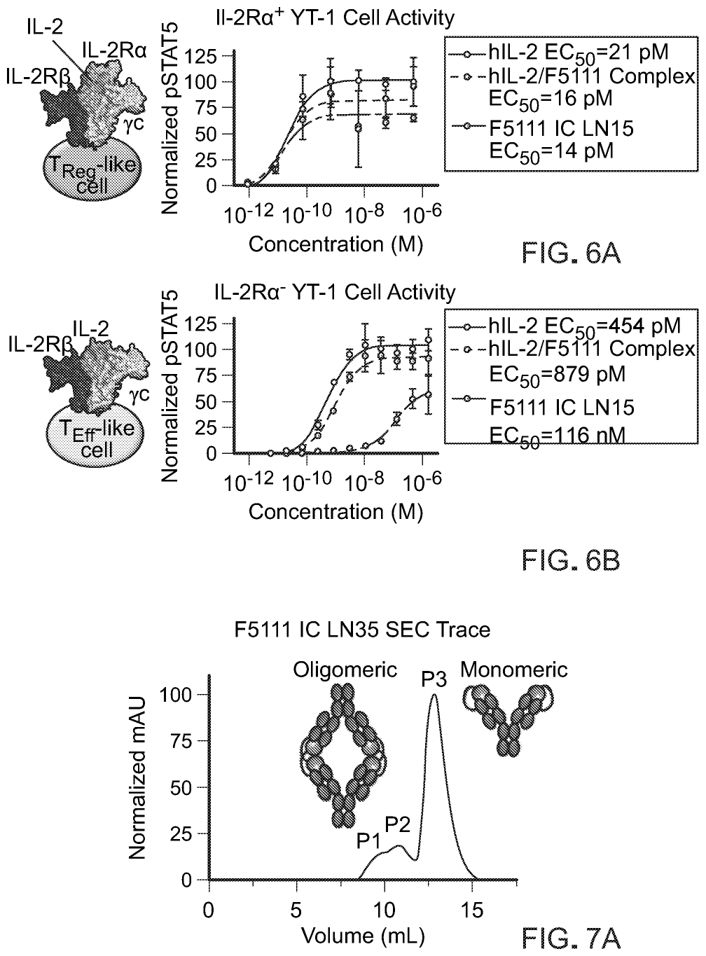

IL-2 signals through activation of signal transducer and activator of transcription 5 (STAT5), which translocates to the nucleus to effect transcriptional programs (see, e.g., Murray, P.J. J Immunol, 178(5): 2623-2629 (2007); and Bromberg, J., and Wang, T.C., Cancer Cell, 15(2): 79-80 (2009)). To characterize IC variant-mediated immune bias, the YT-1 human NK cell line, which inducibly expresses the IL-2Rα subunit was employed (see, e.g., Yodoi et al., J Immunol, 134(3): 1623-1630 (1985)). Flow cytometry-based studies were performed to quantify STAT5 signaling elicited by IL-2, the IL-2/F5111 complex, and F5111 IC LN15 on induced IL-2Rα⁺ versus uninduced IL-2Rα⁻ YT-1 cells as a surrogate for T_{Reg} versus immune effector cell activation (FIG. 6). Untethered IL-2 signals potently on both IL-2Rαa⁺and IL-2Rα⁻cells, and IL-2/F5111 complex fully activated both IL-2Rα⁺ cells and IL-2Rα⁻ cells. In contrast, F5111 IC LN15 activity was only mildly impaired on IL-2Rα⁺cells (FIG. 6A), but its activity was dramatically reduced on IL-2Rα⁻ cells (FIG. 6B).

These results indicate that the F5111 immunocytokine effectively biases IL-2 activity toward T_{Reg} cells over immune effector cells, and it does so significantly more effectively than the mixed IL-2/F5111 complex (FIG. 6).

### EXAMPLE 4

This example describes experiments to optimize expression and function of the F5111 immunocytokine.

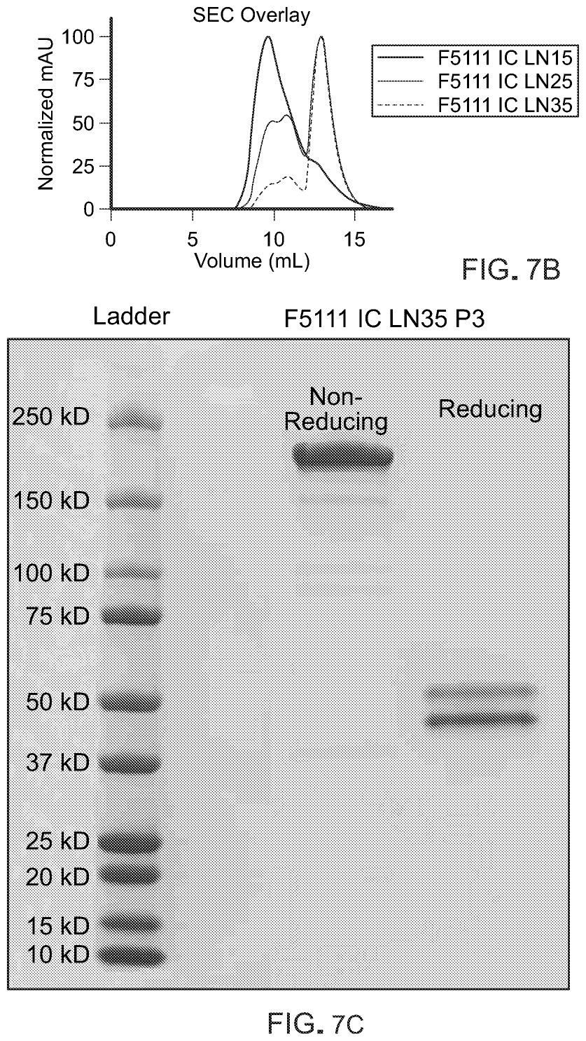

The previously described F5111 IC LN15 includes a 15-amino acid flexible linker between the C-terminus of hIL-2 and the N-terminus of the light chain of the F5111 antibody. Alternative F5111 IC constructs were designed substituting the 15-amino acid linker with longer linkers, including a 25-amino acid linker (F5111 IC LN25) and a 35-amino acid linker (F5111 IC LN35). Expression of F5111 IC LN25 and LN35 was carried out in HEK 293F cells via transient co-transfection of plasmids encoding the F5111 heavy chain and the IL-2-fused F5111 light chain. The protein was purified from cell supernatants via protein G affinity chromatography followed by SEC on a fast protein liquid chromatography (FPLC) instrument. Three peaks were observed by SEC analysis for both F5111 IC LN25 and LN35 (labeled P1, P2, P3), and each peak was pooled for analysis. It was expected that since P1 and P2 elute earlier from the SEC column, they contain higher order oligomeric structures, whereas P3 represents the monomeric F5111 IC LN35 (FIG. 7A). The SEC traces for F5111 IC LN25 and LN35 were compared to the SEC trace for F5111 IC LN15 (FIG. 7B). As seen in FIG. 7B, most of the produced the F5111 IC LN15 was oligomerized, demonstrated by its coincident elution with P1 and P2 of F5111 IC LN25 and LN35 from the SEC column. Furthermore, P3 of F5111 IC LN25 and LN35 eluted at a volume close to the molecular weight of an antibody, suggesting that this peak consists of the monomeric IC. Therefore, P3 was used for evaluation in further experiments with F5111 IC LN25 and LN35, and all subsequent references to F5111 IC LN25 and LN35 represent P3 unless otherwise specified. F5111 IC LN35 contained overall less oligomer compared to F5111 IC LN25. SDS-PAGE analysis was performed to verify purity (FIG. 7C).

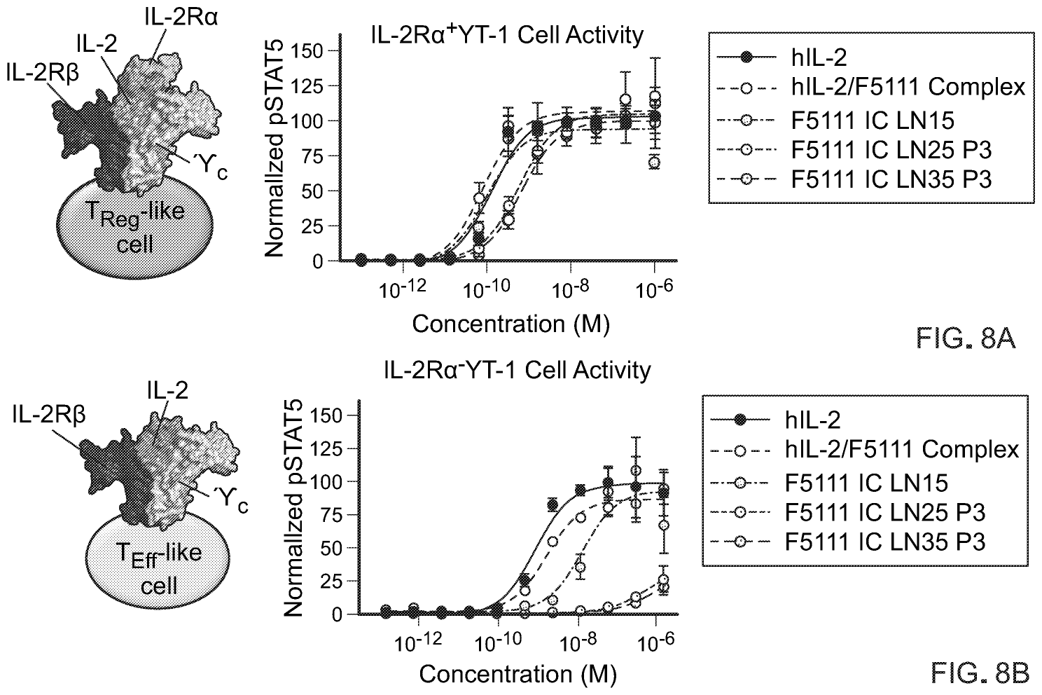

It was demonstrated that F5111 IC LN25 and LN35 selectively activate IL-2Rα⁺T_{Reg}-like cells over IL-2Ra″ T effector (TEff)-like cells with greater bias than F5111 IC LN15 and the hIL-2/F5111 complex. A cell signaling assay was performed on YT-1 human NK cells with or without IL-2Rα expression. FIGS. 8A and 8B show STATS phosphorylation in response to hIL-2, hIL-2/F5111 complex, F5111 IC LN15, F5111 IC LN25, or F5111 IC LN35 on IL-2Rα⁺ cells (FIG. 8A) or IL-2Rα⁻ cells (FIG. 8B), as measured by flow cytometry. F5111 IC LN25 and LN35 activated IL-2Rα⁺ cells at sub-nanomolar concentrations, whereas the activity of F5111 IC LN25 and LN35 on IL-2Rα⁻ cells was immeasurably weak, demonstrating the bias of these ICs toward IL-2Rα-expressing T_{Reg}-like cells.

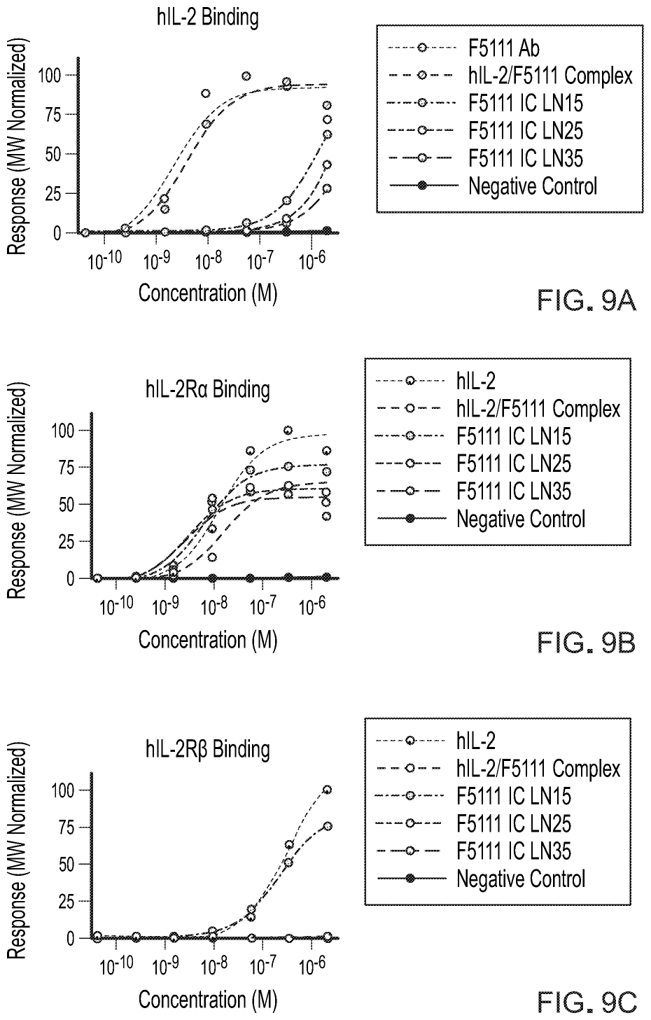

Experiments were conducted to demonstrate that hIL-2 is intramolecularly bound to the antibody within the F5111 IC LN25 and LN35 constructs, and that the F5111 IC LN25 and LN35 selectively direct hIL-2 to T_{Reg} cells by fully blocking the IL-2Rβ binding site to favor interaction with cells that express high levels of IL-2Rα. Binding interactions between IL-2 and F5111 IC LN25 and LN35 were evaluated using bio-layer interferometry on an Octet® instrument with biotinylated hIL-2 immobilized to streptavidin-coated tips (FIG. 9A). Additionally, binding interactions between F5111 IC LN25 and LN35 with the human IL-2Rα (hIL-2Rα) and hIL-2Rβ subunits were measured using bio-layer interferometry on an Octet® instrument by immobilizing biotinylated hIL-2Rα and IL-2Rβ on streptavidin-coated tips. As shown in FIG. 9B, F5111 IC LN25 and LN35 had similar binding affinities toward hIL-2Rα compared to free hIL-2, hIL-2/F5111 complex, and F5111 IC LN15. In contrast, there was a significant reduction in the binding affinity to hIL-2Rβ for the F5111 IC LN25 and LN35 compared to free hIL-2, hIL-2/F5111 complex, and F5111 IC LN15, further illustrating the improved molecular bias of F5111 IC LN25 and LN35 compared to F5111 IC LN15, as well as the hIL-2/F5111 complex (FIG. 9C).

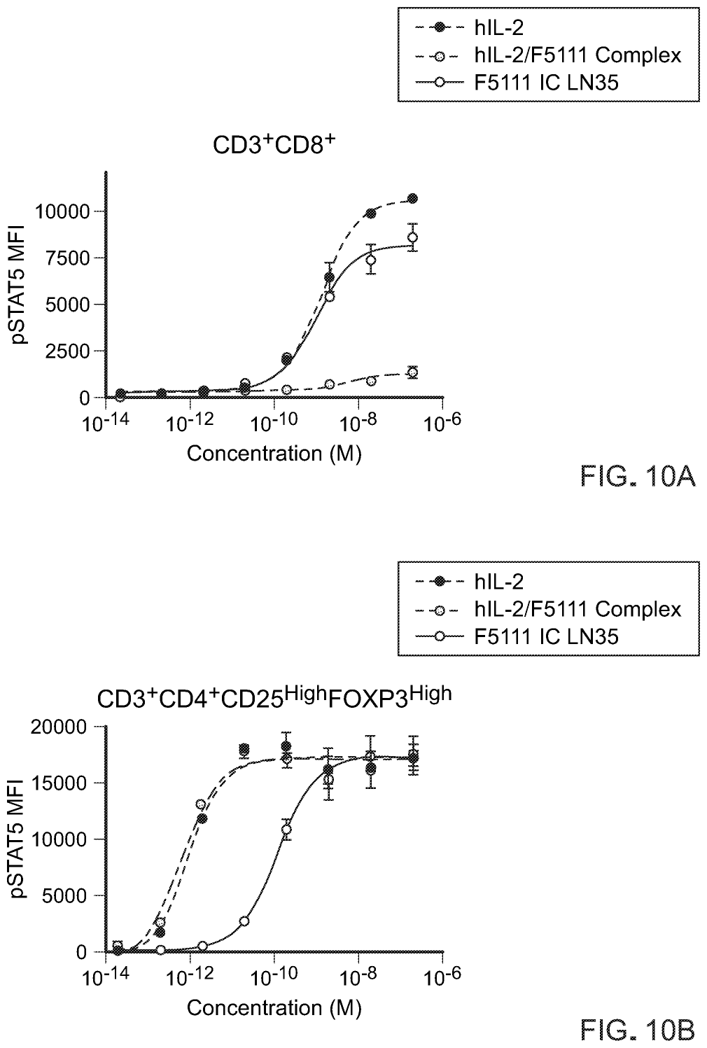

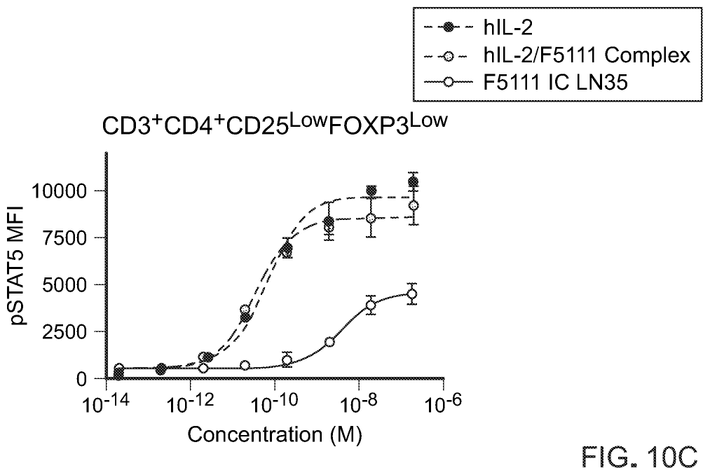

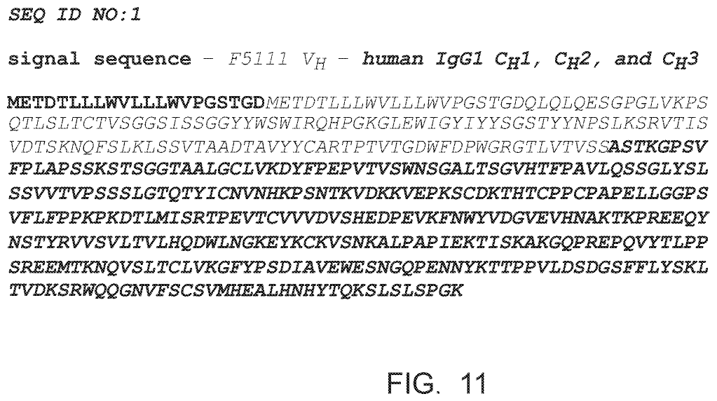

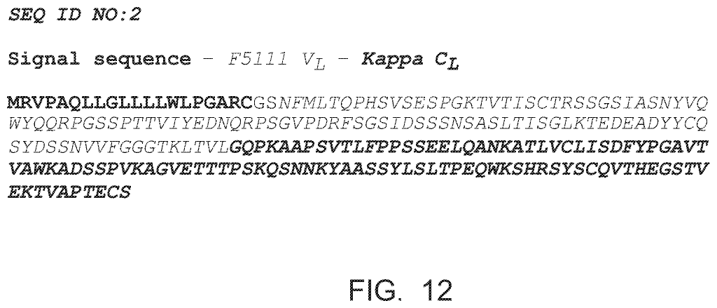

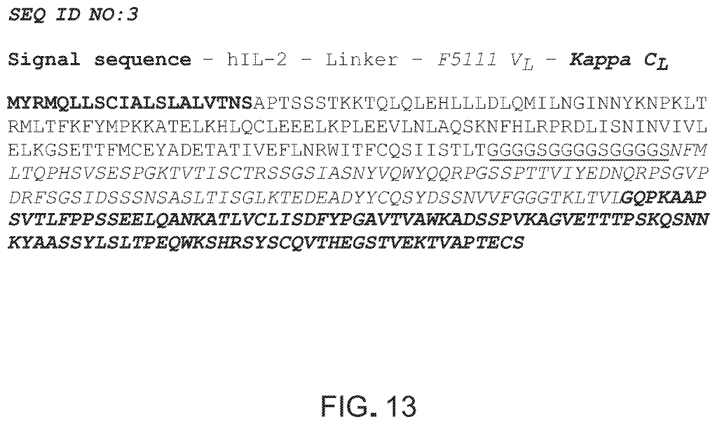

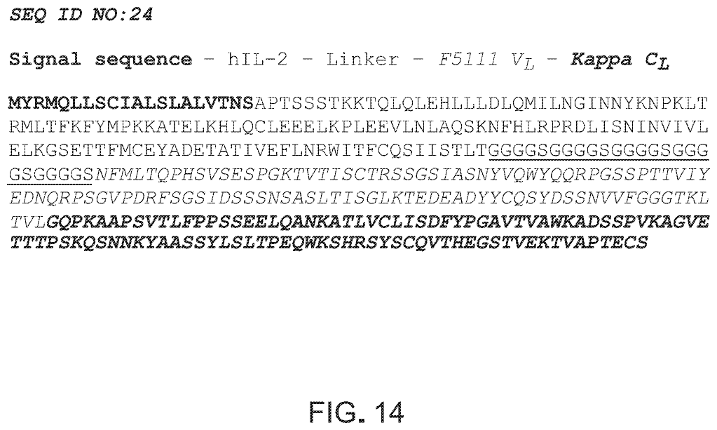

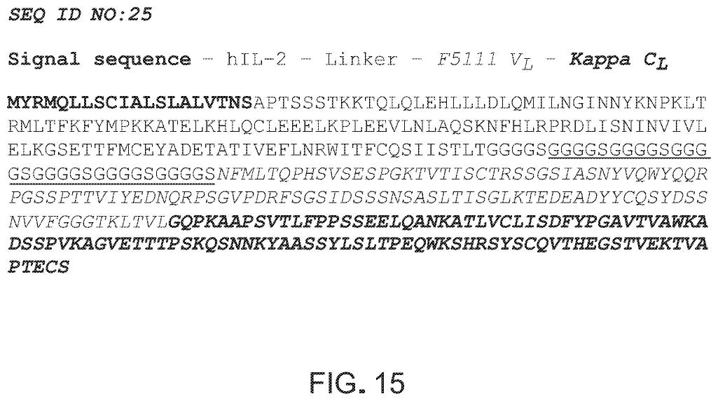

Immunocytokine activity was also interrogated on human PBMCs, isolated from healthy donor whole blood. STATS phosphorylation was measured to quantify activation of 3 cell populations: CD3⁺CD8⁺cells (CD8⁺ effector T cells) (FIG. 10A), CD3⁺CD4⁺CD25^{High}FOXP3^{High} cells (T_{Reg} cells) (FIG. 10B), and CD3⁺CD4⁺CD25^{Low}FOXP3^{Low} cells (CD4⁺effector T cells) (FIG. 10C). F5111 IC LN35 demonstrated significantly more potent activation of T_{Reg} cells compared to both CD8⁺ T cells and CD4⁺ effector T cells. In contrast, hIL-2/F5111 complex treatment showed no T cell subset bias compared to free hIL-2.

Summary

These results demonstrate that IL-2/F5111 immunocytokines can selectively expand T_{RegS}, and can therefore be used to suppress pathogenic autoimmunity and mitigate transplant rejection directly in patients.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

SEQUENCE LISTING FREE TEXT:
Signal sequence - F5111 V_{H} - human IgG1 C_{H}1, C_{H}2, and C_{H}3
SEQ ID NO: 1
METDTLLLWVLLLWVPGSTGDQLQLQESGPGLVKPSQTLSLTCTVSGGSISSGGYYWS
WIRQHPGKGLEWIGYIYYSGSTYYNPSLKSRVTISVDTSKNQFSLKLSSVTAADTAVYYC
ARTPTVTGDWFDPWGRGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYS
KLTVDKSRWQQGNVFSCSMHEALHNHYTQKSLSLSPGK
Signal sequence - Linker - F5111 V_{L} - human Lambda C_{L}
SEQ ID NO: 2
MRVPAQLLGLLLLWLPGARCGSNFMLTQPHSVSESPGKTVTISCTRSSGSIASNYVQW
YQQRPGSSPTTVIYEDNQRPSGVPDRFSGSIDSSSNSASLTISGLKTEDEADYYCQSYDSS
NVVFGGGTKLTVLGQPKAAPSVTLEPPSSEELQANKATLVCLISDFYPGAVTVAWKADSSPVK
AGVETTTPSKQSNNKYAASSYLSLTPEQWKSHRSYSCQVTHEGSTVEKTVAPTECS
Signal sequence - human IL-2 - Linker - F5111 V_{L} - human Lambda C_{L}
SEQ ID NO: 3
MYRMQLLSCIALSLALVTNS

[symbol]

[symbol]

[symbol]

[symbol]

[symbol]

[symbol]

[symbol]

GGGGSGGGGSGGGGSNFMLTQPHSV
SESPGKTVTISCTRSSGSIASNYVQWYQQRPGSSPTTVIYEDNQRPSGVPDRFSGSIDSSSN
SASLTISGLKTEDEADYYCQSYDSSNVVFGGGTKLTVLGQPKAAPSVTLFPPSSEELQANK
ATLVCLISDFYPGAVTVAWKADSSPVKAGVETTTPSKQSNNKYAASSYLSLTPEQWKSHRSYSCQ
VTHEGSTVEKTVAPTECS
F5111 V_{H}
SEQ ID NO: 4
QLQLQESGPGLVKPSQTLSLTCTVSGGSISSGGYYWSWIRQHPGKGLEWIGYIYYSGSTY
YNPSLKSRVTISVDTSKNQFSLKLSSVTAADTAVYYCARTPTVTGDWFDPWGRGTLVTV
SS
Human IgG1 C_{H}1, C_{H}2, and C_{H}3
SEQ ID NO: 5
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
Signal sequence
SEQ ID NO: 6
METDTLLLWVLLLWVPGSTGD
Signal sequence
SEQ ID NO: 7
MRVPAQLLGLLLLWLPGARC
Signal sequence
SEQ ID NO: 8
MYRMQLLSCIALSLALVTNS
Human IL-2
SEQ ID NO: 9
APTSSSTKKTQLQLEHLLLDLQMILNGINNYKNPKLTRMLTFKFYMPKKATELKHLQCLE
EELKPLEEVLNLAQSKNFHLRPRDLISNINVIVLELKGSETTFMCEYADETATIVEFLNRWI
TFCQSIISTLT
F5111 V_{L}
SEQ ID NO: 10
NFMLTQPHSVSESPGKTVTISCTRSSGSIASNYVQWYQQRPGSSPTTVIYEDNQRPSGVPD
RFSGSIDSSSNSASLTISGLKTEDEADYYCQSYDSSNVVFGGGTKLTVL
Human Lambda C_{L}
SEQ ID NO: 11
GQPKAAPSVTLFPPSSEELQANKATLVCLISDFYPGAVTVAWKADSSPVKAGVETTTPSK
QSNNKYAASSYLSLTPEQWKSHRSYSCQVTHEGSTVEKTVAPTECS
Linker
SEQ ID NO: 12
GGGGSGGGGS
Linker
SEQ ID NO: 13
GGGGSGGGGSGGGGS
Linker
SEQ ID NO: 14
GGGGSGGGGSGGGGSGGGGS
Linker
SEQ ID NO: 15
GGGGSGGGGSGGGGSGGGGSGGGGS
Linker
SEQ ID NO: 16
GGGGSGGGGSGGGGSGGGGSGGGGSGGGGS
Linker
SEQ ID NO: 17
GGGGSGGGGSGGGGSGGGGSGGGGSGGGGSGGGGS
Linker
SEQ ID NO: 18
GGGGSGGGGSGGGGSGGGGSGGGGSGGGGSGGGGSGGGGS
Linker
SEQ ID NO: 19
GGGGSGGGGSGGGGSGGGGSGGGGSGGGGSGGGGSGGGGSGGGGS
Linker
SEQ ID NO: 20
GGGGSGGGGSGGGGSGGGGSGGGGSGGGGSGGGGSGGGGSGGGGSGGGGS
Linker
SEQ ID NO: 21
GGGGSGGGGSGGGGSGGGGSGGGGSGGGGSGGGGSGGGGSGGGGSGGGGSGGGGS
Linker
SEQ ID NO: 22
GGGGSGGGGSGGGGSGGGGSGGGGSGGGGSGGGGSGGGGSGGGGSGGGGSGGGGSG
GGGS
Biotin acceptor peptide
SEQ ID NO: 23
LNDIFEAQKIEWHE
Signal sequence - human IL-2 - Linker - F5111 V_{L} - human Lambda C_{L}
SEQ ID NO: 24
MYRMQLLSCIALSLALVTNS

[symbol]

[symbol]

[symbol]

[symbol]

[symbol]

[symbol]

[symbol]

GGGGSGGGGSGGGGSGGGGSGGGGS
NFMLTQPHSVSESPGKTVTISCTRSSGSIASNYVQWYQQRPGSSPTTVIYEDNQRPSGVPD
RFSGSIDSSSNSASLTISGLKTEDEADYYCQSYDSSNVVFGGGTKLTVLGQPKAAPSVTLF
PPSSEELQANKATLVCLISDFYPGAVTVAWKADSSPVKAGVETTTPSKQSNNKYAASSYLSLTPE
QWKSHRSYSCQVTHEGSTVEKTVAPTECS
Signal sequence - human IL-2 - Linker - F5111 V_{L} - human Lambda C_{L}
SEQ ID NO: 25
MYRMQLLSCIALSLALVTNS

[symbol]

[symbol]

[symbol]

[symbol]

[symbol]

[symbol]

[symbol]

GGGGSGGGGSGGGGSGGGGSGGGGS
GGGGSGGGGSNFMLTQPHSVSESPGKTVTISCTRSSGSIASNYVQWYQQRPGSSPTTVIY
EDNQRPSGVPDRFSGSIDSSSNSASLTISGLKTEDEADYYCQSYDSSNVVFGGGTKLTVL
GQPKAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECS

What is claimed is:

1. A single-chain immunocytokine comprising: an immunoglobulin heavy chain; an IL-2 polypeptide, wherein said IL-2 polypeptide can bind to a polypeptide complex comprising an interleukin-2 receptor-α (IL-2Rα) polypeptide, an interleukin-2 receptor-β (IL-2Rβ) polypeptide, and a common gamma chain (γc) polypeptide (an IL-2Rα/IL-2Rβ/γc polypeptide complex); and an immunoglobulin light chain; wherein said single-chain immunocytokine binds to said IL-2Rα/IL-2Rβ/γc polypeptide complex.

2. The single-chain immunocytokine of claim 1, wherein said immunoglobulin heavy chain comprises a variable domain having at least 80% identity to an amino acid sequence set forth in SEQ ID NO:4.

3. The single-chain immunocytokine of claim 2, wherein said immunoglobulin heavy chain comprises a variable domain having an amino acid sequence set forth in SEQ ID NO:4.

4. The single-chain immunocytokine of any one of claims 2-3, wherein said immunoglobulin heavy chain comprises a y heavy chain constant domain.

5. The single-chain immunocytokine of claim 4, wherein said γ heavy chain constant domain has at least 70% identity to an amino acid sequence set forth in SEQ ID NO:5.

6. The single-chain immunocytokine of any one of claims 4-5, wherein said immunoglobulin heavy chain comprises a constant domain having an amino acid sequence set forth in SEQ ID NO:5.

7. The single-chain immunocytokine of any one of claims 2-6, wherein said immunoglobulin heavy chain comprises a signal sequence.

8. The single-chain immunocytokine of claim 7, wherein said signal sequence comprises an amino acid sequence set forth in SEQ ID NO:6.

9. The single-chain immunocytokine of any one of claims 2-8, wherein said immunoglobulin heavy chain comprises an amino acid sequence set forth in SEQ ID NO:1.

10. The single-chain immunocytokine of claim 1, wherein said IL-2 polypeptide comprises an amino acid sequence having at least 80% identity to an amino acid sequence set forth in SEQ ID NO:9.

11. The single-chain immunocytokine of claim 10, wherein said IL-2 polypeptide comprises an amino acid sequence set forth in SEQ ID NO:9.

12. The single-chain immunocytokine of claim 1, wherein said immunoglobulin light chain comprises a variable domain having at least 80% identity to an amino acid sequence set forth in SEQ ID NO: 10.

13. The single-chain immunocytokine of claim 12, wherein said immunoglobulin light chain comprises a variable domain having an amino acid sequence set forth in SEQ ID NO:10.

14. The single-chain immunocytokine of any one of claims 12-13, wherein said immunoglobulin light chain comprises a lambda (λ) light chain constant domain.

15. The single-chain immunocytokine of claim 14, wherein said λ, light chain constant domain has at least 70% identity to an amino acid sequence set forth in SEQ ID NO:11.

16. The single-chain immunocytokine of any one of claims 14-15, wherein said immunoglobulin light chain comprises a constant domain having an amino acid sequence set forth in SEQ ID NO:11.

17. The single-chain immunocytokine of any one of claims 12-16, wherein said immunoglobulin light chain comprises a signal sequence.

18. The single-chain immunocytokine of claim 17, wherein said signal sequence comprises an amino acid sequence set forth in SEQ ID NO:7.

19. The single-chain immunocytokine of any one of claims 12-18, wherein said immunoglobulin light chain comprises an amino acid sequence set forth in SEQ ID NO:2.

20. The single-chain immunocytokine of claim 1, wherein said IL-2 polypeptide and said immunoglobulin light chain are a fusion polypeptide.

21. The single-chain immunocytokine of claim 20, wherein said IL-2 polypeptide comprises an amino acid sequence having at least 80% identity to an amino acid sequence set forth in SEQ ID NO:9.

22. The single-chain immunocytokine of claim 21, wherein said IL-2 polypeptide comprises an amino acid sequence set forth in SEQ ID NO:9.

23. The single-chain immunocytokine of claim 20, wherein said immunoglobulin light chain comprises a variable domain having at least 80% identity to an amino acid sequence set forth in SEQ ID NO: 10.

24. The single-chain immunocytokine of claim 23, wherein said immunoglobulin light chain comprises a variable domain having an amino acid sequence set forth in SEQ ID NO:10.

25. The single-chain immunocytokine of any one of claims 23-24, wherein said immunoglobulin light chain comprises a λ, light chain constant domain.

26. The single-chain immunocytokine of claim 25, wherein said λ, light chain constant domain has at least 70% identity to an amino acid sequence set forth in SEQ ID NO:11.

27. The single-chain immunocytokine of any one of claims 25-26, wherein said immunoglobulin light chain comprises a constant domain having an amino acid sequence set forth in SEQ ID NO:11.

28. The single-chain immunocytokine of any one of claims 20-27, wherein said IL-2 polypeptide and said immunoglobulin light chain are fused via a linker.

29. The single-chain immunocytokine of claim 28, wherein said linker is a peptide linker comprising from 10 to 60 amino acids.

30. The single-chain immunocytokine of claim 29, wherein said linker is a (Gly₄Ser)₃ linker, a (Gly₄Ser)₅, or a (Gly₄Ser)₇.

31. The single-chain immunocytokine of any one of claims 20-30, wherein said immunoglobulin light chain comprises a signal sequence.

32. The single-chain immunocytokine of claim 31, wherein said signal sequence comprises an amino acid sequence set forth in SEQ ID NO:8.

33. The single-chain immunocytokine of any one of claims 20-32, wherein said immunoglobulin light chain comprises an amino acid sequence set forth in SEQ ID NO:3, SEQ ID NO:24, or SEQ ID NO:25.

34. The single-chain immunocytokine of any one of claims 1-33, wherein said single-chain immunocytokine has a half-life of from about 5 minutes to about 6 months.

35. The single-chain immunocytokine of any one of claims 1-33, wherein said single-chain immunocytokine has an affinity for an IL-2Rα polypeptide of from about 10 nM K_{D} to about 1 pM K_{D}.

36. The single-chain immunocytokine of any one of claims 1-33, wherein said single-chain immunocytokine has an affinity for an IL-2R13 polypeptide of greater than about 300 nM K_{D}.

37. The single-chain immunocytokine of any one of claims 1-36, wherein said single-chain immunocytokine binds to a human IL-2Rα/IL 2Rβ/γc polypeptide complex.

38. The single-chain immunocytokine of claim 37, wherein said single-chain immunocytokine does not binds to a non-human IL-2Rα/IL 2Rβ/γc polypeptide complex.

39. A nucleic acid encoding the single-chain immunocytokine of any one of claims 1-38.

40. The nucleic acid of claim 39, said nucleic acid comprising a first nucleic acid and a second nucleic acid, wherein said first nucleic acid can encode said an immunoglobulin heavy chain, and wherein said second nucleic acid can encode said IL-2 polypeptide fused to said immunoglobulin light chain.

41. A method for treating a mammal having an autoimmune disease, said method comprising: administering a composition comprising the single-chain immunocytokine of any one of claims 1-38 or a composition comprising the nucleic acid of any one of claims 39-40 to said mammal.

42. The method of claim 41, wherein said mammal is a human.

43. The method of any one of claims 41-42, where said autoimmune disease is selected from the group consisting of type 1 diabetes, multiple sclerosis, Chron's disease, ulcerative colitis, psoriasis, graft-versus-host disease, Guillain-Barre syndrome, lupus, rheumatoid arthritis, chronic inflammatory demyelinating polyneuropathy, Hashimoto Thyroiditis, Celiac disease, Addison disease, autoimmune hepatitis, antiphospholipid syndrome, and Graves disease.

44. The method of any one of claims 41-43, further comprising administering one or more autoimmune disease treatments to said mammal under conditions wherein number of autoantibodies present in said mammal is reduced.

45. A method for stimulating regulatory T cells in a mammal, said method comprising:
administering a composition comprising the single-chain immunocytokine of any one of claims 1-38 or a composition comprising the nucleic acid of any one of claims 39-40 to said mammal.

46. The method of claim 45, wherein said mammal is a human.

47. A method for treating a mammal having a transplant rejection, said method comprising: administering a composition comprising the single-chain immunocytokine of any one of claims 1-38 or a composition comprising the nucleic acid of any one of claims 39-40 to said mammal.

48. The method of claim 47, wherein said mammal is a human.

49. The method of any one of claims 47-48, wherein said transplant rejection comprises rejection of an allogeneic transplant or an autologous transplant.

50. The method of any one of claims 41-49, wherein said method does not substantially activate effector T cells.
